(19) United States
(12) Patent Application Publication
(10) Pub. No.: US 2009/0118810 A1
(43) Pub. Date: May 7, 2009

(54) STENT ASSEMBLY SYSTEM

(75) Inventors: Richard Klein, Santa Rosa, CA (US); James C. Peacock, III, San Carlos, CA (US)

Correspondence Address: K&L Gates LLP;ATTN: Daniel S. Kim, 1900 MAIN STREET, SUITE 600, IRVINE, CA, 92614-7319, US

(73) Assignee: Medlogics Device Corporation, Santa Rosa, CA (US)

(21) Appl. No.: 12/110,217

(22) Filed: Apr. 25, 2008

Related U.S. Application Data
(63) Continuation-in-part of application No. 10/529,108, filed on Sep. 12, 2005.
(60) Provisional application No. 60/414,177, filed on Sep. 27, 2002.
(60) Provisional application No. 60/459,254, filed on Mar. 30, 2003.

(51) Int. Cl. A61F 2/06; A61B 19/00

(52) U.S. Cl. 623/1.11; 623/1.15; 623/1.12; 623/1.42; 128/898

(57) ABSTRACT

A stent is provided with modified ends (782, 786) and enhanced drug delivery. The stent assembly also provides for enhanced overlapping between adjacent stents.

### CROSS REFERENCE TO RELATED APPLICATIONS

The present application is a continuation-in-part of U.S. patent application Ser. No. 10/529,108 which is a national phase application under 35 U.S.C. §371 of International Application No. PCT/US03/30902 filed on Sep. 29, 2003, which claims the benefit under 35 U.S.C. §119(e) of U.S. Provisional Patent Application Nos. 60/414,177 filed on Sep. 27, 2002 and 60/459,254 filed Mar. 30, 2003, all of which are incorporated by reference herein in their entirety.

### FIELD OF THE INVENTION

This invention is an implantable medical device and related methods of manufacture and use. More specifically, it is an implantable endoluminal stent.

### BACKGROUND OF THE INVENTION

Implantable stents have been under significant development for more than a decade, and many different designs have been investigated and made commercially available for use in providing mechanical scaffolding to hold body lumens open or “patent.” Stents are generally used in many different body lumens, including in particular blood vessels, and more specifically coronary and peripheral arteries. Other body lumens where stents have been disclosed for use include pulmonary veins, gastrointestinal tract, biliary duct, fallopian tubes, and vas deferens. Still further, artificial lumens have been created in the body in a man-made effort to provide artificial communication or transport within the body, such as for example shunts, and transmyocardial revascularization, and stents have been disclosed for intended use in these lumens as well.

Vascular stents are generally tubular members formed from a lattice of structural struts that are interconnected to form an integrated strut network that forms a wall that surrounds an axis. The integrated strut lattice typically includes inter-strut gaps through which the inner lumenal axis within the stent wall and outer region surrounding the stent wall are able to communicate. This is beneficial for example in the setting of stent implantation along a length of a main lumen, e.g. an artery, where side branches may beneficially receive flow from the main lumen through the gaps in the stent wall.

The majority of commercially available stents form completely integrated tubular structures, with continuity found along the integrated strut lattice both circumferentially as well as longitudinally. In order to provide for the adjustability between the collapsed and expanded conditions, such stents generally incorporate undulating shapes for the struts, which shapes are intended to reconfigure to allow for maximized radial expansion with minimized longitudinal change along the stent length. This is generally desirable for example in order to achieve repeatable, predictable placement of the stent along a desired length of localized, diseased region to be re-opened (e.g. occlusion), as well as maintain stent coverage over the expanding balloon at the balloon ends. Else, a stent that substantially shortens during balloon expansion exposes the balloon ends to localized vessel wall trauma at those ends without the benefit of the stent scaffolding to hold those regions open long-term after the intervention is completed.

Notwithstanding the prevalence of the foregoing type of stent just described, other designs have also been disclosed that either further modify such general structures, or further depart from the basic design. For example, one additional type of stent forms a wall that is not circumferentially continuous, but has to opposite ends along a sheet formed from the strut lattice. This sheet is adjusted to the collapsed condition by rolling the stent from one end to the other. At the site of implantation, the stent is unrolled to form the structural wall that radially engages the lumenal wall and substantially around an inner lumen. In the event the stent is undersized to the lumen, the opposite ends overlap and thus double the thickness of implant material that protrudes from the lumen wall and into the lumen.

Stents are most frequently used in an interventional recanalization procedure, adjunctive to methods such as balloon angioplasty, or atherectomy such as rotational atherectomy devices and methods. “Balloon expandable” stents are generally constructed from a material, such as stainless steel or cobalt-chromium alloy for example, that is sufficiently ductile to be delivered in a collapsed condition on an outer surface of a deflated balloon, and is then expandable by inflation of the balloon to an expanded condition against the subject lumenal wall and that is substantially retained in such condition as an implant upon subsequent balloon deflation. “Self-expanding” stents are generally constructed of an elastic, super-elastic or shape-memory material, such as particular metal alloys including for example nickel-titanium alloys. These materials typically have a memory state that is expanded, but is delivered to the implantation site in a collapsed condition for appropriate delivery profiles. Once in place, the stent is released to recover or “self-expand” against the lumenal wall where it is then left as the implant.

Stents are typically intended to maintain patency, other uses have been disclosed. For example, some stents have been disclosed for the purpose of occluding the subject lumen where the stent is implanted. Examples of such stents include fibrin coated stents, and examples of such occlusive uses for stents include fallopian tubal ligation and aneurysm closure.

Stents have been further included in assemblies with other structures, such as grafts to form “stent-grafts”. These assemblies generally incorporate a stent structure that is secured to a graft material, such as formed from a textile or sheet material type construction, Examples of uses that have been disclosed for stent-grafts include for example aneurysm isolation, such as in particular along the abdominal aorta wall.

In the particular setting of vascular stenting, stents have had an enormous impact upon the occurrence of “restenosis” following recanalization procedures. “Restenosis” is a re-occlusion of the acutely recanalized blockage that typically takes place within 3-6 months after intervention, and is generally a combination of mechanical and physiological responses to the vessel wall injury caused by the recanalization procedure itself. In one regard, restenosis can occur at least in part from an elastic recoil of the expanded vessel wall, such as following expansion of the wall during balloon angioplasty. With respect to the physiological response to injury, it has generally been observed that injury from the recanalization to the intimal, medial, and sometimes adventitial layers of a vessel wall causes smooth muscle cells within the wall to undergo aggressive mitosis and hyperproliferation, dividing and migrating into the vessel lumen to form a “scar” that occludes the vessel lumen. Whereas angioplasty and other recanalization interventions prior to the advent of stenting resulted in approximately 30% restenosis rate, stenting has generally reduced this rate to about 20%, which reduction is considered a result of the mechanical prevention of vascular recoil.

Recent efforts in vascular stenting have been intended to incorporate additional therapy adjunctive to stenting to further reduce the incidence of restenosis. Some efforts for example have been intended to locally deliver therapeutic doses of radiation to the vessel wall concomitant to stenting, including for example by incorporating radioactive materials into or on the stent scaffolding itself. However, these efforts carry significant burden peri-operatively in handling and disposing of the materials, and results have yet been considered compelling among the healthcare community. At least one device has been further disclosed to modify certain aspects of the stent ends. However, the proximal end has not been in particular addressed as a location with unique requirements, nor have unique structures been incorporated locally only at the proximal end. Moreover, local energy delivery such as via radioactive stents is substantially different than local elution delivery of materials and compounds from stents which are thereafter subject to diffusion, flow, and other active transport mechanisms.

More recently, a substantial industry effort has been underway to incorporate local drug delivery to stented lesions specifically to retard and prevent restenosis. For example, various local delivery devices have been disclosed to provide highly localized injection of anti-restenosis material into the injured wall, such as via micro-needles incorporated onto the outer skin of expandable balloons.

A more substantial effort, however, has been to incorporate the anti-restenosis drugs on or into the stents themselves in a manner such that the stent elutes the drug into the vessel wall over a prescribed period of time following implantation, otherwise known as drug eluting stents (“DES”). Examples of devices intended for this use include coated stents, which provide a stent structure with an outer coating that holds and elutes the drug. The most prevalent form of these coatings include polymers, such as for example in one particular commercial embodiment a two-layer polymer coating with one layer holding drug and another layer retarding elution to provide extended, or with one layer providing adhesion to the underlying stent metal and the other layer holding and eluting the drug. Other examples of DES coatings include ceramics, hydrogels, biosynthetic materials, and metal-drug matrix coatings. Examples of drugs that have been investigated for anti-restenosis uses such as via DES methods include anti-mitotics, anti-proliferatives, anti-inflammatory, and anti-migratory compounds. Further examples of compounds previously disclosed for use in DES devices and methods include: angiotensin converting enzyme (ACE) inhibitors, angiotensin receptor antagonists, anti-sense materials, anti-thrombotics, platelet aggregation inhibitors, iron chelators (e.g. exochelin), everolimus, tacrolimus, vasodilators, nitric oxide, and nitric oxide promoters or donors.

Two more specific compounds that have been under substantial clinical investigation on DES devices include Rapamycin™ (sirolimus) and Taxol™ (paclitaxel). These DES efforts have made substantial strides toward reducing restenosis rates from the typical rate in stented lesions of about 20%, to a reduced rate around 10%, and possibly lower in particular with respect to certain patient sub-populations.

Notwithstanding these substantial improvements that appear to be anticipated in view of the recent sirolimus and paclitaxel DES clinical experiences, however, various needs still remain and are believed to be unmet by these and other previously disclosed DES efforts.

In one regard, those DES clinical experiences generally include stent implants with substantially similar design to prior uncoated stents—there is little if any modification or optimization provided to those stent designs to enhance drug delivery. However, the drug delivery provided by the stents is dictated by the strut shape and overall lattice design which carries the eluting coating.

It is therefore generally believed that the anticipated 10% restenosis rate expected with these particular DES devices may be further improved by modifying the drug delivery platform with improved stents designed to meet delivery requirements of potent drugs in addition to the prior design parameters, which in the past had been driven principally by mechanical considerations to providing a structural scaffolding.

More specifically, certain previously disclosed DES clinical trial results and related analysis have identified that the ends of stents have been associated with localized regions of increased restenosis within this nevertheless reduced pool of patients suffering from restenosis. Moreover, closer inspection of such clinical data reveals that such association between restenosis and stent ends is further related to multiple, respectively unique situations and considerations as follows.

In one regard, the ends of single implanted stents (e.g. non-overlapping), and more specifically the arterial “segment” adjacent the ends of the stents, have been associated with localized incidence of restenosis. Stents are often implanted in a slightly “oversized” configuration versus the underlying vessel, such that they are expanded to slightly larger than the main lumen diameter adjacent the lesion site. Therefore, these mechanical structures, typically constructed of stainless steel, cobalt-chromium, or other strong metals, result in abrupt transitions with adjacent, unstented vessel wall. In addition, according to at least one DES disclosure, elution of these typically highly hydrophobic drugs has been shown to remain localized in tissue adjacent to the stent struts themselves. To the extent that injury is done adjacent to but beyond the ends of stents, such injury is not receiving the benefit of drug delivery from the struts as is experienced within the longitudinal confines of the implant.

Still further, the proximal (or upstream) ends of the DES implants are further observed to present higher incidence of restenosis than the distal end. However, the proximal ends of stents, including in particular the stents used in published DES clinical trials, are constructed from the same design as the distal ends of the stents. These stent ends are generally defined at the apices or crowns of undulating cycles of shaped strut lattice, whereas an apex shape is believed to produce a structure that forms a tendency to remain in the plain of the bend and thus increases stiffness against a bending moment at the vessel-stent transition. As the artery wall is in constant motion, this transition is believed to be a site of inflammatory interaction with potential erosion results over time. Moreover, to the extent that any transport mechanisms do exist along a vessel wall that may effect drug migration and thus tissue delivery kinetics from the eluting stent struts, such is generally believed to follow a “downstream” direction, e.g. with the flow of the vessel, and further with respect to vaso vasorem within a vessel wall itself.

Accordingly, it is believed that the vessel wall tissue immediately adjacent and upstream from the proximal end of a typical DES stent implant represents a location with potentially the most extensive injury, but the least anti-restenosis drug delivery. By increasing the drug elution dose from the stent struts, diffusion may provide for the necessary treatment efficacy at such region. However, previously disclosed DES efforts provide a constant dose along the stent, and harm may result from overdosing the subject drugs, many of which are toxic at certain levels. To provide the dose necessary to “reach” the upstream tissue via diffusion would potentially provide too much drug along the stent, with possible harm including tissue necrosis and possibly aneurysms resulting from negative remodeling around the “high dose” stent.

Prior disclosures have included stents with modified ends to meet certain particular intended goals provided in those disclosures, such as for example localized radiopaque markers. However, these disclosures have yet to address the unique biomechanical requirements located at the proximal, upstream end of the stent. For example, several prior disclosures provide for a unique design to the ends of the stent, but both ends are implicated with the same design in these cases. However, there are considerations that must be taken into account for the design at the distal end of a stent, including in particular the ability to provide a minimized collapsed profile on that distal shoulder for initially crossing tight lesions. Designs according to such considerations need not apply to the proximal end of the stent, but such has not been heretofore given its proper specific considerations.

There is therefore a need for an improved DES device and method that provides one or more enhanced design parameters specifically along the proximal end of the device that is unique relative to and differs from the distal end of the stent and also relative to the mid-body of the stent.

There is still a particular need for an improved DES device and method that provides for enhanced drug delivery along the proximal end of the stent that differs from the distal end of the stent and from the mid-body of the stent.

There is also still a particular need for an improved stent with a proximal end that minimizes trauma, inflammation, and/or erosion at the tissue-device interface along the upstream edge of the stent.

By providing a unique design along the proximal edge of the stent, much improvement may be accomplished to meet these needs at the expense of other considerations, such as for example profile, that plays a much reduced role and concern at this very different location during in-vivo use. Moreover, if such can be accomplished without expensing profile, then even more benefit may be provided, either at the proximal edge location, or by allowing such beneficial features to be incorporated at the distal end.

According to recent DES clinical trial reports, another location where specifically increased incidence of restenosis appears to still remain within the patient pool, also implicating the ends of stents, is directly related to “overlapping” stents. “Overlapping stents” are generally herein defined as multiple stents that are implanted in series along a length of vessel and are “overlapped”. This overlapping occurs after a first stent is in place, whereas the following second stent is expanded within the first one in overlapping fashion to provide continuity to one long stented region along the vessel. Many practitioners desire such overlap for adjacent stents to provide such continuity, and typically target for example about 5 millimeters of overlap (e.g. between the proximal end one stent and the overlapping distal end of the other stent) in the implanted result. Others attempt to minimize the extent of overlap as much as possible, and target about 1 mm to about 5 mm of length for overlapping. Few practitioners specifically attempt to avoid such stent overlap, however, based upon the belief that such overlap causes more harm than benefit.

More specifically, the combination, overlapping lattice structure that results from overlapping two opposing stents is in particular undesirable within a blood pool, such as in an artery, where poor fluid dynamics along the stented wall are compounded by the overlapped region, possibly resulting in an increased risk of thrombogenesis in that area. Moreover, thrombogenesis along an injured vessel wall has further been reported as a “pressor” to the hyperproliferation of smooth muscle cells, and thus a pressor to restenosis.

According to certain published DES clinical trial results, it is believed that the clinical incidence of restenosis in overlapping stents has been significantly reduced by the respective DES devices and methods used. However, at least one such published study has indicated that restenosis at the overlapping zone between overlapping stents still occurs with particular increased frequency within the overall DES-treated patient population. More specifically, in this study, over half of the clinical restenosis observed in the trial population were focal lesions exactly at the area of overlap between overlapping stents.

Notwithstanding the foregoing observations, there has yet to be a stent or combination stent assembly that is specifically designed to improve and enhance the biocompatibility and long-term efficacy, and more specific restenosis, associated with overlapping stents in particular.

There is therefore still a need for an implantable vascular stent that is particularly adapted to overlap with another stent and to improve the long term efficacy, and in particular reduce the thrombogenic and restenotic response, associate with such overlapping.

### SUMMARY OF THE INVENTION

Disclosed herein is a stent assembly system comprising two or more stents allowing for control of drug elution in the overlapping portions of the stents.

In one embodiment, a stent assembly for implanting at least two stents at a location within a lumen is provided comprising first and second delivery systems each having a proximal end and a distal end that is adapted to be positioned at a location within a lumen; first and second stents each with a first end portion, a second end portion and a central portion disposed between the respective first and second end portions; the first end portion, the second end portion and the central portion defining a longitudinal axis along the length of each of the stents; each of the first end portions having a first lattice structure that is different than a central lattice structure along the corresponding central portion; the first stent is mounted on the distal end of the first delivery system with the first end portion located proximally of the second end portion; the second stent is mounted on the distal end of the second delivery system with the first end portion located distally of the second end portion; each of the first and second stents having a radially collapsed condition for delivery to the location; wherein at the location each of the first and second stents are adjustable from the respective radially collapsed condition to a radially expanded condition.

In another embodiment, when expanded at the location, at least a part of the first end portions overlap.

In another embodiment, the first lattice structures of each of the first end portions further have a lattice structure that is different than a second lattice structure along the corresponding second end portion. In another embodiment, the first lattice structure comprises a first circumferentially undulating pattern with a plurality of first strut segments wherein a circumferential array of M first end crowns is formed between adjacent first strut segments with the first undulating pattern having a first amplitude; the second lattice structure comprises a second circumferentially undulating pattern with a plurality of second strut segments wherein the circumferential array of N second end crowns is formed between adjacent second strut segments with the second undulating pattern having a second amplitude; and wherein said first and second amplitudes are different. In one embodiment, M is less than N. In another embodiment, M is 0.5 N.

In another embodiment, the stent assembly further comprises a bioactive agent in association with the first and the second stent wherein each of the first and second stents is adapted to exhibit a gradient of varied bioactive agent elution profiles along the respective stent length. In one embodiment, the gradient of varied bioactive agent elution profiles along the stent length comprises a first elution profile along the first end portion, a second elution profile along the central portion; and a third elution profile along the second end portion that is different than the first and second elution profiles.

In yet another embodiment, when the stent assembly is expanded at the location, at least a part of the first end portions overlap to create an overlap zone and wherein the elution profile at the overlap zone is less than double the elution profile along the remaining portions of the stented segment. In another embodiment, the struts and crowns of the first end portion are of reduced thickness as compared to the struts and crowns of the second end portion or said central portion.

In another embodiment, the stent assembly further comprises a bioactive agent in association with the first stent and the second stent; the respective first end portions are adapted to elute the bioactive agent according to a first elution profile; the respective second end portions are adapted to elute the bioactive agent according to a second elution profile; the respective central portions are adapted to elute the bioactive agent according to a third elution profile; and wherein the first elution profile is substantially less than either the second or third elution profiles. In another embodiment, the second and third elution profiles are substantially equivalent.

In one embodiment, the first end, second end and central portions have the same longitudinal length. In another embodiment, the central portions have a longitudinal length that is greater than the longitudinal length of the first and the second end portions. In another embodiment, the first end portion has a longitudinal length that is about twice the longitudinal length of either of the second end portion or the central portion.

In another embodiment of the stent assembly, the first end portion comprises one or more elements and wherein the total longitudinal length of the first end portion does not exceed 4.0 mm. In another embodiment, each first end portion comprises up to four elements. In another embodiment, each element independently has a longitudinal length from about 0.5 mm to about 3.0 mm.

In yet another embodiment of the stent assembly, the first delivery system further includes a marker band at one or more locations selected from the group consisting of proximal to said first end portion, distal to said first end portion and distal to said second end portion. In another embodiment, the second delivery system further includes a marker band at one or more locations selected from the group consisting of distal to said first end portion, proximal to said first end portion and proximal to said second end portion.

In another embodiment, a method for stenting a wall at a location within a lumen in a body of a patient is provided comprising delivering a first stent to a first implant location within the lumen; delivering a second stent to a second implant location within the lumen that overlaps with the first implant location such that confronting ends of the first and second stents overlap wherein the confronting end of each of the first and second stents comprise a lattice structure that is different than the lattice along the remaining portion of the respective stent.

In an embodiment the first and second steps overlap in a manner such that their region of overlap does not have a substantially increased thickness profile of the wall of the lumen at the location. In another embodiment, the longitudinal length of the overlap does not exceed 50% of the longitudinal length of either of the first stent or the second stent. In another embodiment, the longitudinal length of said overlap is about 3.0 mm to about 5.0 mm.

In another embodiment, a method for stenting a wall at a location within a lumen in a body of a patient is provided comprising delivering a first stent to a first implant location within the lumen; delivering a second stent to a second implant location within the lumen that overlaps with the first implant location such that confronting ends of the first and second stents overlap at an overlap zone and eluting a bioactive agent from the first and second stents such that an elution profile at the overlap zone is less than double an elution profile along the remaining portions of the stented segment.

Further objects and advantages of the invention will be brought out in the following portions of the specification, wherein the detailed description is for the purpose of fully disclosing preferred embodiments of the invention without placing limitations thereon.

### BRIEF DESCRIPTION OF THE DRAWINGS

The invention will be more fully understood by reference to the following drawings which are for illustrative purposes only:

FIG. 1A depicts a partial exploded side view of one end portion of a stent in a collapsed condition according to one embodiment.

FIG. 1B depicts a partially exploded side view of a similar stent shown in FIG. 1A, except in an expanded condition.

FIG. 1C depicts a further exploded view of more detail of certain features of the strut and crown arrangement at the end of a stent according to the embodiment shown in FIGS. 1A-B.

FIG. 2 shows another exploded view of an end crown and related strut configuration according to another embodiment.

FIG. 3 shows another exploded view of an end crown and strut configuration according to another embodiment.

FIG. 4A shows an end view of a primary shape of a stent body for reference.

FIG. 4B shows a side view of a secondary shape of a stent body for reference.

FIG. 5 shows a side view of a tertiary shape of a stent body according to one embodiment and incorporated with the primary and secondary shapes shown in FIGS. 4A-B.

FIGS. 6A-C show various side views of a stent end according to another embodiment and according to different modes and levels of detail, respectively.

FIG. 7 shows another view similar to FIG. 6C, except according to another embodiment.

FIGS. 8A-B show a side view of another stent end according to another embodiment, and show the stent end in collapsed and expanded configurations, respectively.

FIG. 9 shows a side view of another stent end according to another embodiment.

FIG. 10 shows a side view of another stent end according to another embodiment.

FIG. 11 shows a side view of another stent end portion and adjacent stent body according to another embodiment of the invention that includes a sleeve at the stent end.

FIG. 12 shows a side view of another stent end according to another embodiment.

FIGS. 13A-B show side views of two stent end portions according to two further embodiments, respectively.

FIGS. 14A-C show various levels of detail and respective operating conditions of a strut and crown arrangement according to another embodiment.

FIGS. 15A-B show various aspects of an overlapping stent system according to another aspect.

FIGS. 16A-B show certain cross-sectional detail of various struts at particular locations along the overlapping stents shown in FIG. 15A-B, respectively.

FIG. 16C show certain cross-sectional detail related to various modes of overlapping struts shown in FIGS. 16A-B.

FIGS. 17A-B show certain further aspects of the overlapping stent system shown in FIGS. 15A-B as the stents are incorporated onto balloon expansion delivery systems, respectively.

FIGS. 18A-C shows various a schematic graphs illustrating certain drug elution profile gradients according to certain features of various embodiments.

FIGS. 19A-B show schematic cross-sections of two drug eluting stent strut configurations according to two further embodiments, respectively.

FIG. 20A shows a plan view of one stent according to the invention in a mode after the stent is cut longitudinally and laid flat in a plane, and shows a first end of the stent having a unique local end-crown structure versus the other opposite end.

FIG. 20B shows a similar view to that shown in FIG. 20A of another stent embodiment with the respective structures on the first and second end portions interchanged relative to the embodiment shown in FIG. 20A.

FIG. 21A shows a similar view to that shown in FIG. 20B of another stent embodiment with another unique localized end-crown structure and configuration on the second end that is modified from the embodiment shown in FIG. 20B.

FIG. 21B shows an end view of the second end of the stent shown in FIG. 21A in one mode of a “rolled-down” radially collapsed configuration with overlapping end-crowns.

FIG. 22 shows a similar view to that shown in FIG. 21A of another stent embodiment with unique local end-crown structures on the second end that are modified from the embodiment shown in FIG. 21A.

FIG. 23A shows a similar view to that shown in FIG. 22 of another stent embodiment with unique local end-crown structures on the second end that are modified from the embodiment shown in FIG. 22.

FIG. 23B shows an end view of the second end of the stent shown in FIG. 23A in one mode of a “rolled-down” radially collapsed configuration with overlapping end-crowns.

FIG. 24A shows a similar view to that shown in FIG. 23A of another stent embodiment with unique local end-crown structures on the second end that are modified from the embodiment shown in FIG. 23.

FIG. 24B shows an end view of the second end of the stent shown in FIG. 24A in one mode of a “rolled-down” radially collapsed configuration with overlapping end-crowns.

FIG. 25A shows a similar view to that shown in FIG. 24A of another stent embodiment with unique local end-crown structures on the second end that are modified from the embodiment shown in FIG. 24A.

FIG. 25B shows an end view of the second end of the stent shown in FIG. 25A in one mode of a “rolled-down” radially collapsed configuration with overlapping end-crowns.

FIG. 26A shows a similar view to that shown in FIG. 25A of another stent embodiment with unique local structures on the second stent end that are modified from the embodiment shown in FIG. 25A.

FIG. 26B shows an end view of the second end of the stent shown in FIG. 26A in one mode of a “rolled-down” radially collapsed configuration with overlapping end-crowns.

FIG. 27 shows a similar view to that shown in FIG. 26 of another stent embodiment with multiple, unique local structures along two adjacent stent segments along a first end portion of the stent that are unique to that end portion relative to the rest of the stent and different than the other opposite end portion of the stent.

FIG. 28A shows a similar view to that shown in FIG. 27 of another stent embodiment with the multiple, unique local structures located at the two adjacent stent segments along the first end portion of the stent similar to those shown in the FIG. 27 embodiment, but with reduced thickness for the strut scaffolding along those segments versus the corresponding thicknesses located elsewhere along the stent.

FIG. 28B shows a similar view to that shown in FIG. 28A of another stent embodiment with the respective structures at the first and second end portions interchanged relative to the embodiment shown in FIG. 28A, and with a modified interconnecting arrangement between stent segments along the body of the stent relative to the embodiment shown in FIG. 28A.

FIG. 29 shows a similar view to that shown in FIG. 28B of another stent embodiment with other unique local structures located at the two adjacent strut segments along the second end portion of the stent that are modified relative to the embodiment shown in FIG. 28B, and with an interconnecting arrangement between stent segments along the stent body similar to the embodiment shown in FIG. 28A.

FIG. 30A shows a similar view to that shown in FIG. 28B of another stent embodiment with a modified shape along the stent end crowns along the second end portion versus the shape shown in FIG. 28B.

FIG. 30B shows an end view of the second end of the stent shown in FIG. 30A in one mode of a “rolled-down” radially collapsed configuration with overlapping end-crowns.

FIG. 31A shows a plan view of another overlapping stent embodiment in a mode after the stent is cut longitudinally and laid flat in a plane, and shows the first and second ends of the stent having unique local structures relative to each other and also relative to the body portion of the stent.

FIG. 31B shows an end view of the first end portion of the stent shown in FIG. 26A in one mode of a “rolled-down” radially collapsed configuration with overlapping end-crowns.

FIG. 32A shows a view similar to that shown in FIG. 31A of another overlapping stent embodiment.

FIG. 32B shows an end view of the first end portion of the stent shown in FIG. 32A in one mode of a “rolled-down” radially collapsed configuration with overlapping end-crowns.

FIG. 33A shows a similar view to that shown in FIG. 32A of another overlapping stent embodiment.

FIG. 33B shows an end view of the second end of the stent shown in FIG. 33A in one mode of a “rolled-down” radially collapsed configuration with overlapping end-crowns.

FIG. 34A shows another similar view to that shown in FIG. 33A of another overlapping stent embodiment.

FIG. 34B shows an end view of the second end of the stent shown in FIG. 34A in one mode of a “rolled-down” radially collapsed configuration with overlapping end-crowns.

FIG. 35A shows another similar view to that shown in FIG. 34A of another overlapping stent embodiment.

FIG. 35B shows an end view of the second end of the stent shown in FIG. 35A in one mode of a “rolled-down” radially collapsed configuration with overlapping end-crowns.

FIG. 36 shows a plan view of two exemplary, conventional stents in overlapping arrangement.

FIG. 37 shows a similar view to that shown in FIG. 36 of one exemplary conventional stent similar to one of the stents shown in FIG. 36 in overlapping arrangement with an overlapping stent according to a further embodiment.

FIG. 38 shows a similar view to that shown in FIG. 37, except showing two overlapping stents in overlapping arrangement according to still a further embodiment.

FIG. 39 shows an SEM image of a side view along an end portion of a stent manufactured according to certain of the present embodiments with two alternating circumferential arrays of end-crowns having different sized bulb-shaped ends along the stent's end portion.

FIG. 40 shows a picture of an end perspective view of a stent manufactured with a circumferential array of crowns with enlarged bulb-shaped enlargements.

FIG. 41 shows a picture of two overlapping stents manufactured and assembled onto balloon catheters similar to the embodiments shown in FIGS. 17A and B.

FIG. 42 shows a picture taken at 20× magnification under light microscopy of two overlapping stents in overlapping arrangement according to certain embodiments.

FIG. 43 shows a picture taken at 20× magnification under light microscopy of two commercially available stents in overlapping arrangement.

FIG. 44 shows a picture of two commercially available stents in overlapping arrangement, which picture is overlaid onto a graphical illustration of a drug elution profile expected from such overlapping arrangement in the context of adapting the overlapping stents for drug elution in conventional fashion.

FIG. 45 shows a picture of two overlapping stents in overlapping arrangement according to one embodiment, and shows the picture overlaid onto a graphical illustration of a drug elution profile expected from such overlapping arrangement in the context of adapting the particular overlapping stents shown for drug elution also in conventional fashion.

FIG. 46A-E depicts embodiments of first end portions of stents, each first end portion comprising one or more elements of varying length.

FIG. 47A-D depicts stent embodiments in the distal (FIGS. 47A and C) and proximal (FIGS. 47B and D) arrangements and in both collapsed (FIGS. 47A and B) and expanded (FIGS. 47C and D) configurations.

FIG. 48 depicts two stents implanted into a body lumen wherein the distal portion of the first stent overlaps the proximal portion of the second stent.

### DETAILED DESCRIPTION OF THE INVENTION

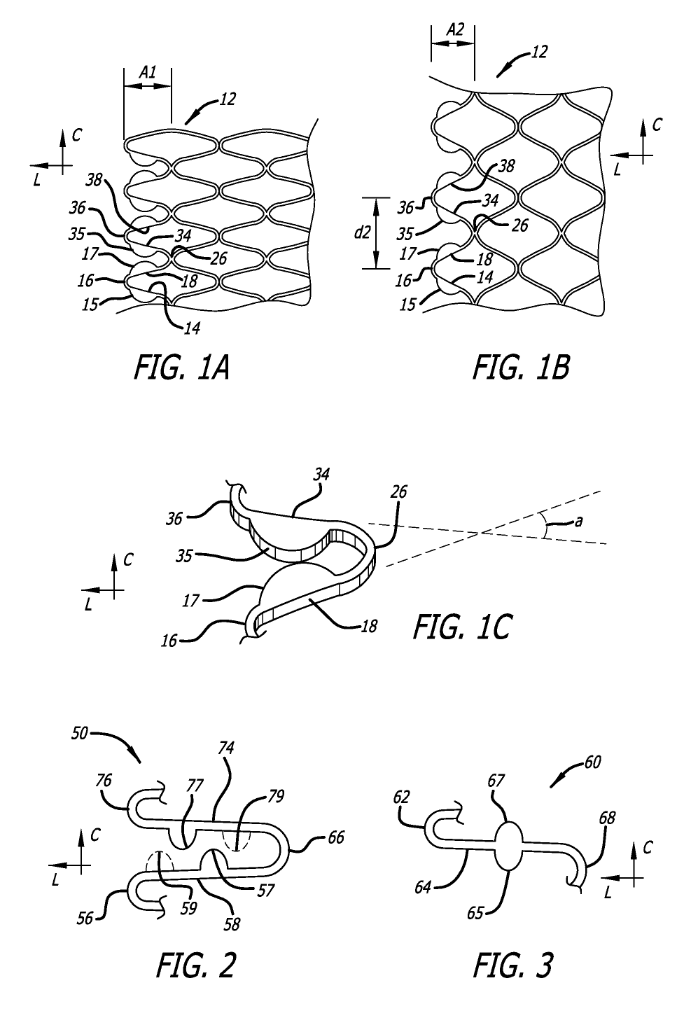

Referring more specifically to the drawings, for illustrative purposes the stent assembly system is embodied in the apparatus generally shown in FIG. 1A through FIG. 48. In general, it is to be appreciated that the various embodiments provide enhancements for improved outcomes for drug eluting stents, wherein a stent scaffolding is adapted to carry and elute bioactive agents. Such may be provided for example in or as a coating along surfaces of the stent scaffolding, or in wells or cavities formed along the stent scaffolding, that hold and elute the drugs. These various enhancements are not intended to be limited to a particular modality of such drug carrying mode, and other modes than those specifically described are contemplated as would be apparent to one of ordinary skill based upon review of this disclosure in view of other available information related to drug eluting stents. It is further appreciated that, despite the various benefits afforded by the present enhancements to drug eluting stents, various such features provide substantial benefit to stent chasses notwithstanding the presence or absence of drug elution, and should be further considered as beneficial in other such settings, such as incorporated with and enhancing bare metal stent scaffolds.

It will be appreciated that the apparatus may vary as to configuration and as to details of the parts, and that the method may vary as to the specific steps and sequence, without departing from the basic concepts as disclosed herein. As shown variously in the figures, various stent embodiments are provided with a tubular wall shown for purpose of illustration in a “splayed open” configuration according to a cut formed longitudinally along the tube along a longitudinal axis L. Accordingly, a circumference of the stent is shown in flat orientation along a circumferential axis C that is transverse to the longitudinal axis L. Further to the various figures showing this arrangement, the top side of the flat illustrations for the stent wall would be generally brought together with the bottom of such flat illustrations in order to form the circumferential wall of the stent along longitudinal axis L, at which time the circumferential axis C shown to be transverse to longitudinal axis L would be modified to fold circumferentially around longitudinal axis L, whereas a radial axis R (not shown) would replace circumferential axis C as the axis being transverse to longitudinal axis L.

It is to be appreciated that certain figures show an entire view of the stent according to this arrangement, or may show partial views in this configuration, as would be apparent to one of ordinary skill for each figure. Such structures may be formed in such a flat “splayed open” manner and then formed into final annular stent products, such as cutting or etching them from flat sheets. Or, they may be formed from rings welded or otherwise secured together from ring segment to ring segment along the longitudinal axis L, or may be cut or etched from a tubular precursor material such as a solid hypotube. Such forming techniques may use laser cutting, photo etching, mechanical cutting, stamping, chemical etching, etc., as would be apparent to one of ordinary skill.

With particular reference to FIGS. 1A-C, these views show various details of one embodiment wherein a stent 10 has certain enhanced features along an end portion 12 as follows. As shown in FIG. 1A, end portion 12 includes a plurality of adjacent end crowns relative to radial axis R that are junction regions formed between adjacent converging strut segments, as shown for illustration at end crown 16 between a pair of converging strut segments 14,18, respectively, or adjacent end crown 36 between converging pair of strut segments 34,38, respectively. These end crowns are essentially alternating peaks of an undulating pattern of a strut segment at the end of the stent 10 that face the same direction relative to longitudinal axis L, as shown for illustration at adjacent end crowns 16 and 36. According to the serpentine pattern shown along radial axis R, the distance d1 between adjacent end crowns, such as between end crowns 16 and 36, thus represents a complete 360 degree cycle of the undulating serpentine pattern at the end portion 12. The distance along the radial axis R between one of the end crowns and the next peak pointing in the opposite direction toward the middle of the stent, such as between end crown 16 and opposite pointing crown 26, represents a 180 degree (or one-half) portion of the serpentine cycle along axis R. The distance between these adjoining crowns 16,26 represents an amplitude of the serpentine pattern for the end portion 12, shown in FIG. 1A in the collapsed configuration as amplitude A1.

It is to be appreciated that this particular type of undulating cycle and specific serpentine shape is chosen for illustration purposes, and, though highly beneficial, is not intended to be limiting to certain broad aspects that are applicable to other strut and crown patterns as would be apparent to one of ordinary skill.

The stent 10 in FIG. 1A includes a plurality of discrete enlargements along the outer surfaces of converging pairs of adjacent stent strut segments forming the end crowns, as shown by reference at enlargements 15,17 along strut segments 14,18 in FIGS. 1A-B. Conversely, these are located along facing surfaces of diverging strut segments in the direction of end 12 that form alternating, adjacent end crowns. To further illustrate in another regard, these enlargements 15,17 are on facing surfaces of converging strut segments within the valley that forms inward facing body crown 26. Each of these descriptions describe the same arrangement from different points of view, and in any case the enlargements provide for an increased amount of drug to be carried at the end portion 12.

These enlargements may be at various locations. In the particular embodiment shown, by carrying them on the strut segments between adjacent end crowns as shown, the impact of the enlargements on stiffness at the stent ends is reduced. More specifically, in the strut/crown configuration exemplified by the present embodiment, the majority of flexure experienced during expansion from the radially collapsed configuration shown in FIG. 1A and the radially expanded configuration shown in FIG. 1B takes place principally at the crowns. Conversely, the strut segments extending between the crowns principally rotate relative to the circumferential axis C about the circumference during expansion. Accordingly, the circumference is expanded from FIG. 1A to FIG. 1B by expanding the distance between crowns, as illustrated by comparing d2 in FIG. 1B with d1 in FIG. 1A. Such radial expansion further results in reducing the amplitude of the undulating cycle as measured between opposite facing adjacent crowns, such as amplitude A2 (FIG. 1B) and A1 (FIG. 1A) between opposite facing crowns 16 and 26. This angle about which the strut segments rotate, and related to the radius of curvature at respective crowns, is shown in finer detail in FIG. 1C by reference to an angle a between strut segments 18 and 34 that converge to form body crown 26, or conversely diverge to form in part adjacent end crowns 16,36. By expanding angle a in adjusting the stent from the collapsed configuration of FIG. 1A to the expanded configuration of FIG. 1B, the facing enlargements 17,35 on struts 18,34, respectively, pull away from each other relative to circumferential axis C, but are positioned to provide increased drug delivery into the expanded gaps between adjacent crowns 16,36 across the distance d2, as shown in FIG. 1B.

It is to be appreciated that various parameters may vary, such as frequency, size, geometry, or location of the strut/crown segments, as well as the enlargements relative to the struts and crowns.

For example, referring to the exploded view of a stent segment 50 in FIG. 2, enlargements 57,77 are located on facing sides of struts 58,74 that converge to form body crown 66, but diverge in the opposite direction to form in part end crowns 56, 76, respectively. These enlargements are at slightly different locations along longitudinal axis L within the amplitude A of the undulating cycle, such that in the collapsed condition shown they do not overlap. In addition, they may be moved to locations 59,79, respectively on struts 58,74. Or, multiple enlargements may be provided, such as at 57 and 59 on strut 58, and enlargements 77 and 79 along strut 74, in a keyed arrangement such that each enlargement fits within an area between struts 58,74 that does not correspond with an opposite facing enlargement.

It is to be appreciated that enlargements may be positioned in other arrangements, including directly facing each other as shown in FIGS. 1A-C, and in that setting may either overlap in the collapsed condition or the collapsed condition can be limited so that they don't reach each other along the circumferential axis C, as shown in FIG. 1A. In addition, enlargements may be provided on either side of a strut, such as shown at enlargements 65,67 on opposite facing sides of strut 64 between opposite facing crowns 62,68 of stent end portion 60 shown in FIG. 3. While the inward facing enlargement 67 is not located so as to “close the gap” between end crown 62 and the next adjacent end crown (not shown) in the expanded configuration, it nevertheless helps to provide still more drug in the general region at the end portion 60 of the corresponding stent.

Other embodiments provide varied shapes of the strut/crown pattern along a stent end portion in order to enhance performance in that region, either with respect to increasing drug delivery there, or impacting stiffness.

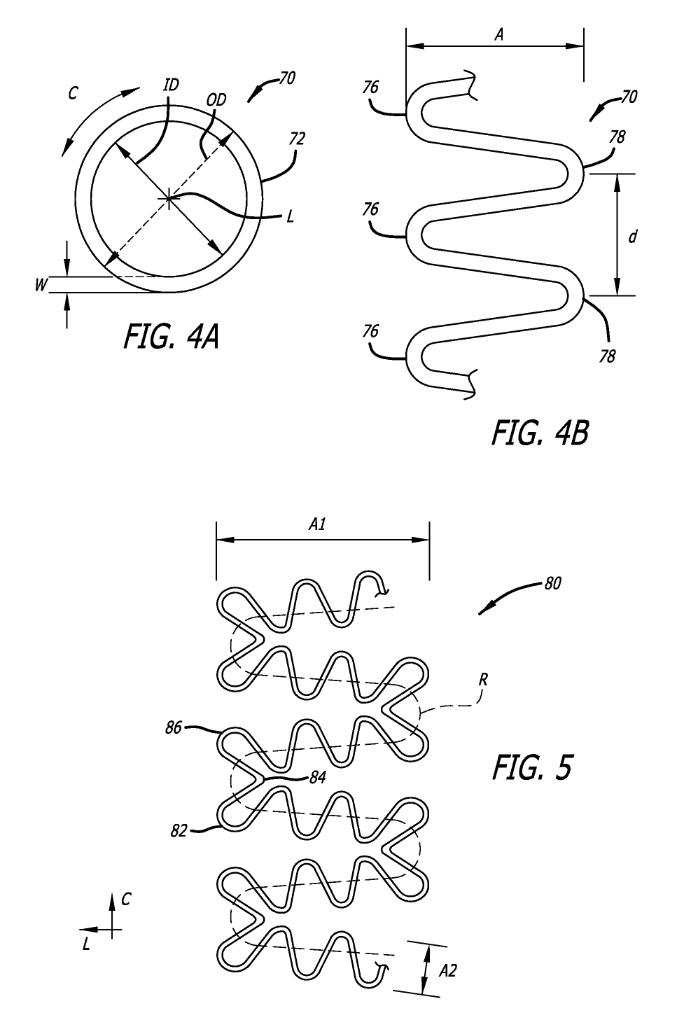

For example, FIGS. 4A-B show end and side views, respectively, of a conventional stent 70 for reference purposes. FIG. 4A illustrates a primary shape for a stent 70 that forms a circumferential wall 72 around a longitudinal axis L with a circumference C and a wall thickness defined by the stent scaffolding thickness between an inner diameter ID and an outer diameter OD. FIG. 4B shows a secondary shape related to the primary shape, wherein the annular wall is formed by a scaffolding which, along the circumferential axis C, has an undulating serpentine pattern with a frequency (number of cycles) between crowns 76 over the circumference, amplitude A between opposite facing crowns 76,78, and distance d designating the circumferential distance of one full cycle between end or body crowns (assuming a symmetric shape at the end and body crown regions of the end portion).

According to one particular embodiment shown in FIG. 5, a tertiary shape is provided along end portion 80 of a stent. This tertiary shape shown includes further undulations along a reference axis R (shown in dashed line) that designates the secondary shape for the pattern. The embodiment shown this tertiary pattern is similar to the secondary pattern, but along a difference reference axis, and includes adjacent tertiary end crowns 82,86 forming a complete cycle of the tertiary pattern, with an opposite facing tertiary body crown 84 therebetween with an amplitude A2 across the reference axis R. This provides for densification of metal, and thus drug, within the end portion 80. This tertiary shape also may provide more flexibility in the end portion 80, and in further embodiments the strut dimensions may be reduced to provide for the denser pattern of drug delivery struts per unit surface area of lumen corresponding to the end portion 80, though with equal or possibly reduced stiffness (or more flexibility) at the stent end portion 80 than would result without the tertiary undulating shape.

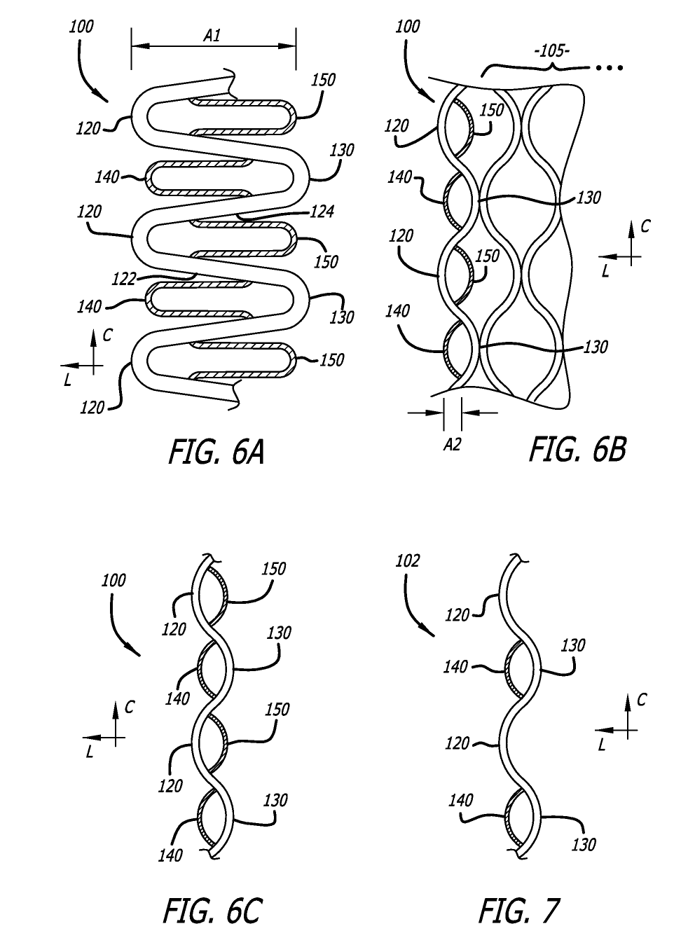

Other shape modifications are contemplated to enhance stent performance at the stent's end portions. For example, FIGS. 6A-C show various side views of a stent end portion 100 according to another embodiment that provide secondary crowns 140,150 that form “reverse undulations” within the various valleys formed between adjacent facing struts between end crowns and body crowns, such as is indicated for reference at reverse undulation 150 between struts 122,124 that converge at end crown 120, or conversely diverge to form in part inward facing adjacent body crowns 130. Such modified stent end portion 100 is shown in radially collapsed configuration in FIG. 6A, and expanded in FIGS. 6B-C, whereas FIG. 6B shows the end portion 100 in context of adjoining body portion 105 and indicates the reduced amplitude A2 of end portion 100 with the modified crown structure in the expanded configuration.

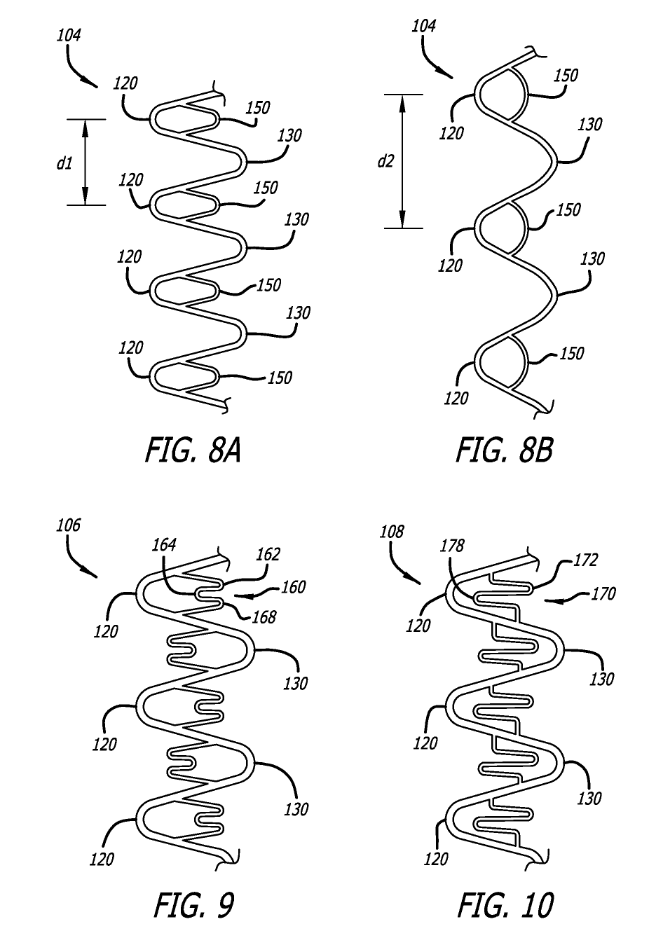

Similar to other embodiments providing discrete modified structures to stent end portions, such reverse undulations may be at each valley of the undulating pattern of the primary serpentine shape as shown in FIGS. 6A-C, or may be at only certain areas, such as only in the valleys between end crowns 120 as shown at secondary end crowns 140 in FIG. 7. This configuration utilizes the particular utility of these reverse undulations to “fill the gap” between the primary end crowns 120 in the expanded configuration, beneficially providing a more continuous drug elution pattern along circumference C where restenosis-causing injury may be present. For the purpose of illustration, FIGS. 8-9 show another embodiment for a stent end portion 104 with another pattern of reverse undulations 150 located within the valleys opposite (or on the body-side of) end crowns 120. While this arrangement does not “fill the gaps” between the end crowns, it does however provide increased surface area for more drug delivery in the general circumferential region of the stent end portion 104. For still further illustration of the diverse variety of patterns contemplated, FIGS. 10A-B show stent end portions 106,108, respectively, with varied forms of secondary or “reverse” undulations 160,170, respectively. As shown in FIG. 10A, reverse undulation 160 includes a secondary shape at its own peak 164 which is inverted yet again on itself to provide a series of mini-crowns 162,164,168 to replace the single reverse undulation crown structure of the immediately preceding embodiments. This provides for yet a further densified pattern of stent scaffold for drug delivery, and furthermore providing for increased flexibility as well as radiopacity. For further illustration of the wide assortment of shapes and patterns contemplated, FIG. 10B shows a further arrangement for providing secondary crowns 170 with a further densified pattern of mini-crowns 172,174.

As further illustrated variously throughout the different embodiments of FIGS. 6A-10B, the secondary or “reverse undulation” crowns of these embodiments may be beneficially constructed from members of thinner thickness than the primary crowns in order to provide for optimized flexibility notwithstanding the addition of more metal in the region. Or, by including such secondary crown structures, the primary crowns may be provided with reduced thickness and still provide similar radial strength against vessel recoil post-implant. This arrangement may be in particular beneficial to provide a better vessel-stent transition at the end crowns.

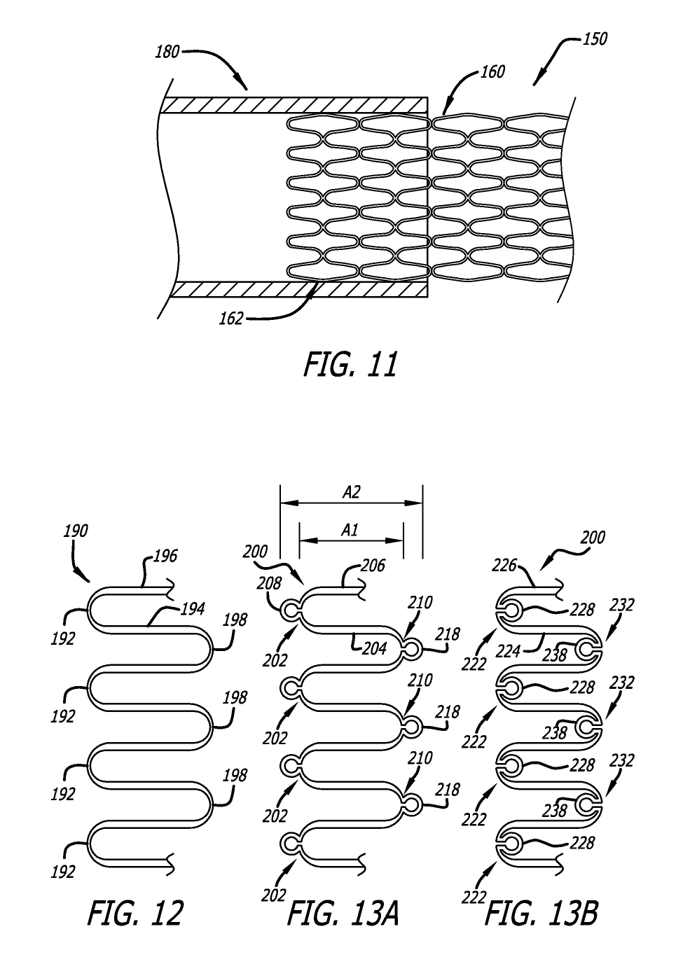

Another embodiment shown in FIG. 11 shows a sleeve 180 at the end portion 160 of stent 150. This sleeve is adapted to provide increased drug elution there, and may extend beyond the stent end portion 160 as shown, or may terminate at the last strut segment along the end crowns (not shown). The sleeve 180 may be on the outside of the stent end 160 as shown, or on the inside (not shown), and can be constructed according to such materials and coupled to the stent 150 according to generally known available options in the art.

Other embodiments not shown load more drug or different release profile at the stent ends with or without the further modifications of the other embodiments herein shown and described, in any event providing for a varied gradient of drug elution along the stent's length as would be apparent to one of ordinary skill, and in particular providing such variation at the stent ends, and still more particularly at the proximal end portion where restenosis rates are at times observed to be highest. One example includes thinner strut scaffold material at the ends, but with more coating and/or drug over those end segments. Another example modifies release formulation of the coating at the end. One variation of this for example uses one coating in the mid or body section of the stent, and a second different coating at the ends. The difference may be different coating all together, or different formulation of a similar coating (e.g. varying 2-part polymer coating for different releases). Another example increases the amount or concentration of the drug itself. Also, a different drug may be incorporated to elute from the end versus the mid or body portion of the stent.

In one particular further embodiment shown in FIG. 12, a stent end portion 190 has varied dimensions along the stent scaffold such that the strut portions, e.g. at struts 194,196, are thicker than at the crown portions 192,198. This capitalizes on the ability to increase thickness, and thus drug elution, along the strut portions that experience very little deformation during stent expansion, whereas the focal regions of deformation at the crowns 192,198 are not compromised. The difference in thickness may be in the underlying scaffold material itself, or may be thicker coating or drug on the strut portions connecting the crowns. In any event, these illustrate yet a further technique herein contemplated to customize features at the stent end portions in order to achieve maximum advantage in one area, e.g. increased drug elution in the stent end portion, while minimizing the sacrifice in another area, e.g. end crown stiffness.

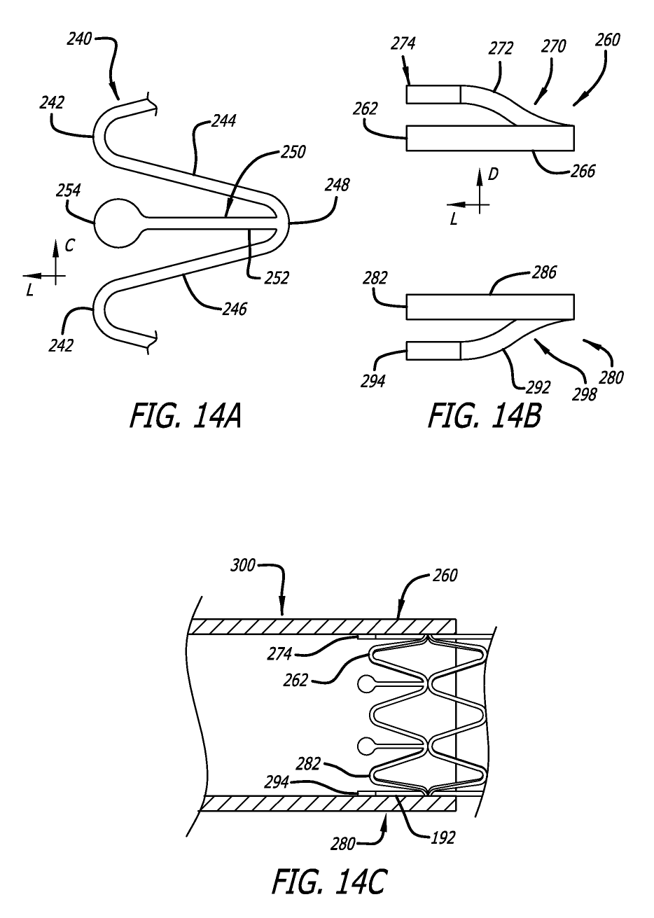

For further illustration, FIGS. 13A-B show still further embodiments of stent end portions 200,220, respectively, with modified scaffold shapes and patterns to increase surface area for drug elution at the end crowns 202,222, respectively, without substantially increasing stiffness during expansion or within the vessel as an implant. More specifically, as apparent to one of ordinary skill in the figures, FIG. 13A provides partially circular bulb enlargements 208 extending beyond the end crowns 202, such as shown at crown 202 formed at the apex between strut segments 204,206 in the FIG. 13A embodiment. FIG. 13B shows a further embodiment where such enlargements modified as enlargements 228 that are invaginated at the respective end crowns 222 inwardly into the valley formed by the strut segments forming the end crowns, such as shown between strut segments 224,226. FIGS. 14A-D variously illustrate another embodiment wherein strut extensions are provided in a manner intended to provide more drug carrying members and thus coverage for drug elution at the stent ends (or elsewhere where incorporated). As shown in FIG. 14A, an enlargement 254 at the end of the extension 250 helps with increased drug elution and minimizes trauma due to the shape of the enlargement 254 (preventing otherwise risk of puncture to a vessel wall). FIG. 14B shows a variation where extensions 270,290 have a spring bias toward an outer periphery of a stent to prevent vessel intrusion when expanded and implanted. The disk-shaped enlargements 274,294, respectively, are gently held flush against the wall of a vessel 300 extending at or beyond the end of the stent, as shown in FIG. 14C. This arrangement helps to deliver drug beyond the end of the stent region where more injury may exist, such as for example due to balloon expansion there or otherwise (e.g. dissections, chronic inflammation, etc.).

For purpose of providing further clarity of illustration, further detail of these present embodiments are provided by reference to FIGS. 14A-C as follows. More specifically, FIG. 14A shows an exploded region of a stent end portion 240 that provides a longitudinal extension 250 with a shank 252 and a radial enlargement or elution disk or pad 254. In the particular embodiment shown, shank 252 extends into the valley between struts 244,246 such that the pad 254 is positioned within the gap between end crowns 242. FIG. 14B shows a side view of two opposite sides of an overall stent structure with two outwardly biased extensions 270,290 biased in opposite directions to correspond with a vessel wall at that area at the implant site. More specifically, extension 270 includes a shank 272 and disk enlargement 274 oriented relative to strut 266 and end crown 262. Extension 290 includes a shank 292 and disk enlargement 294 oriented relative to strut 286 and end crown 282. FIG. 14C shows these features of FIG. 14B in the context of other integrated components of the stent end portion as implanted along a vessel wall 300.

It is to be appreciated therefore according to the foregoing embodiments that stent design directly impacts the drug delivery in drug eluting stents (DES). The stent strut scaffolding is the carrier of the drugs for “non-covered” DES products. Beneficial patterns of designs are thus herein provided to increase drug coverage along a stented wall. In particular, restenosis is still occurring with highest frequency at the stent ends. Published clinical trials with “real world” populations consistently indicate that “in-segment” restenosis (e.g. including 5 mm of vessel adjacent the stent ends) is higher versus “in-stent” restenosis between the stent ends. By localizing novel aspects of stent design at the ends, more drug can be delivered there. Such designs can be used throughout the stent body, but may impact other mechanics in certain circumstances and may be in particular thus most beneficial when provided only at the stent ends (or including closely adjacent scaffolding structures) while leaving the rest of the stent constructed according to a more conventional design.

The foregoing embodiments contemplate incorporation with any and all suitable stent materials and scaffolding designs, and coatings and drugs, in combination with the embodiments shown and described. For example, the additional drug carrying structures shown and described may be integral with the stent, or may use different materials or structures, such as for example different polymers, bioerodable or biodegradable structures, reservoirs formed within stents scaffold, etc. Obvious modifications or improvements therefore made to the particular embodiments herein shown and described, which are generally provided for illustration of certain broad aspects of the invention, are thus contemplated.

In one particular regard, further embodiments are contemplated that provide modified stent ends that are in particular well adapted to improve outcomes related to overlapping multiple stents. More specifically, restenosis has been observed to occur at increased rates at regions of stent overlap in lesions receiving multiple overlapping stents. This has in particular been the case regarding certain clinical trial results for certain DES products. Various embodiments are thus contemplated that provide “overlapping stent” assemblies specially designed for overlapping with other stents in a manner intended to reduce restenosis.

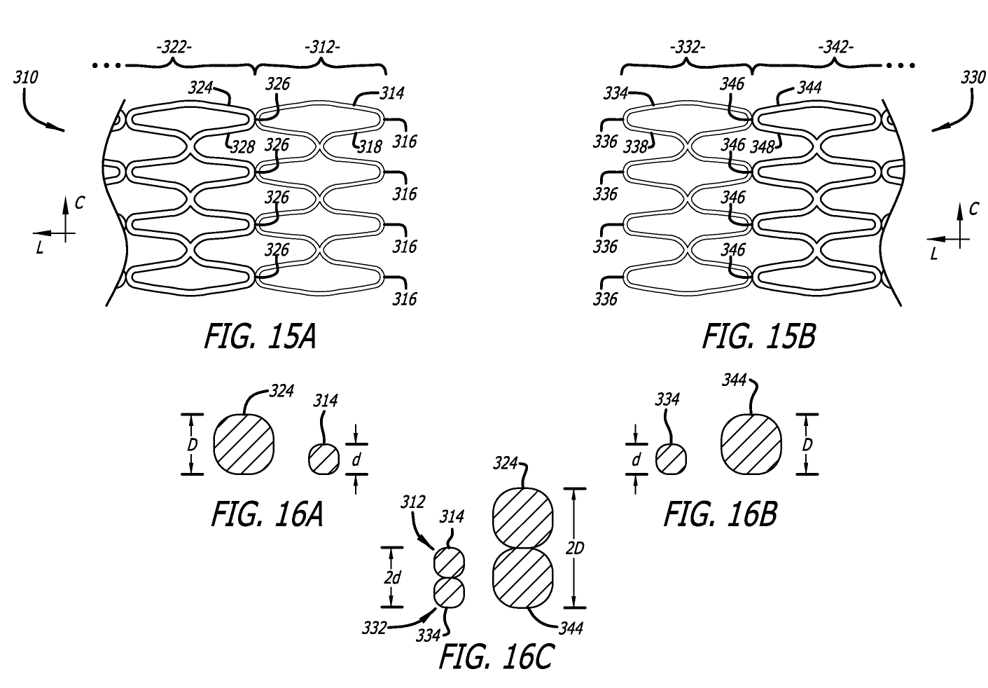

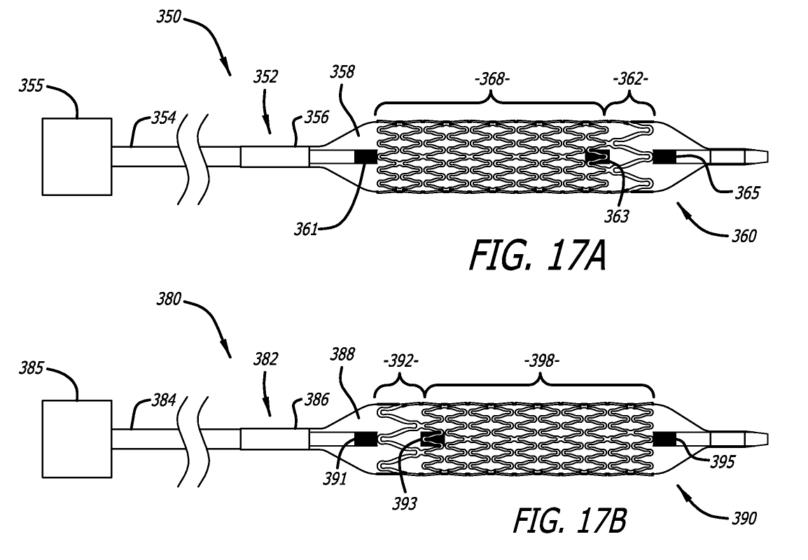

In particular reference to FIGS. 15A-F, these figures thus show various aspects of an overlapping stent system according to another aspect of the invention. More specifically, FIG. 15A shows an exploded view of one overlapping stent 310 with an overlapping end portion 312 having a different design parameter than body portion 322. End crowns 316 and related strut segments, such as shown at struts 314,318, are provided with thinner thickness than the corresponding structures such as at crowns 326 and strut segments, e.g. segments 324,328. This is shown for illustration in FIG. 16A with cross-section of strut 324 shown to have a diameter D and cross-section of strut 314 of the end portion 312 having a diameter d that is about one-half D. In the particular embodiment shown, this end portion 312 comprises two strut scaffold segments, and but for the difference in thickness, the overall design of the undulating patter with respect to amplitude, frequency of crowns, pitch, etc. is similar between the body and end portions 322,312 respectively.

Another similar stent 330 is shown in FIG. 15B in reverse orientation relative to a longitudinal axis L such that the end portion 332 extending from body portion 342 has the thinner structure for end crowns 336 and related strut segments 334,338 facing in the opposite direction versus such segment of stent 310 in the FIG. 15A embodiment. This stent 330 also has a similar modification of strut scaffold thickness as stent 310, as illustrated in comparing body strut 344 having a cross-sectional diameter of D in FIG. 16A with the cross-sectional diameter d of end portion strut 334 in FIG. 16B.

Accordingly, as shown in FIG. 16C, by overlapping the end portion 312 of stent 310 with the end portion 332 of stent 330, the resulting overlap between struts 314 and 334 results in an overall effective thickness at the overlap zone that is equal to 2d, or effectively D. This thus provides a constant diameter along a stented vessel segment of D to the extent that stents 310 and 330 overlap only at the overlapping end zones 312,332, respectively. This compares to an effective doubling of stent thickness of the overlapped region in a more conventional setting, as shown for reference further in FIG. 16C if body struts 324 and 344 were overlapped to yield a 2D combined thickness. Hence, according to the benefits of the present embodiment, the profile of overlap is reduced from 2D to D, considered to provide enhanced hemodynamics along a vessel wall in a flowing artery with reduced shear thrombogenicity. Because thrombogenicity and platelet adhesion is a known precursor to restenosis, this is reasonably anticipated to reduce restenosis outcomes for this reason alone. In addition, stiffness in the overlapped region is reduced also precipitously as the overlapping ends are more flexible to begin with.

As further illustrated in FIG. 17A-B, the overlapping stents just described may be incorporated into an overall kit adapted to provide customized overlapping as follows. Stent delivery systems 350 and 380 each include proximal and distal overlapping stents 360,390, respectively, on balloon catheter delivery systems 352,382, also respectively.

More specifically, balloon catheter 352 shown in FIG. 17A includes an elongate body with a distal end portion 356 that carries an expandable balloon 358 and that is adapted to be positioned within a first location for stent implantation while proximal end portion 354 extends externally from the patient with a coupler 355 adapted to couple to a pressurizable source of inflation fluid, such as an indeflator filled with radiopaque contrast inflation fluid. Proximal overlapping stent 360 is loaded onto balloon 358 with an overlapping end portion 362 located distally of the rest of the stent 368 that includes the stent body and the opposite, proximal stent end portion. The stent assembly system depicted in FIG. 17A also includes marker bands 361,363,365.

FIG. 17B shows balloon catheter 382 that includes an elongate body with a distal end portion 386 that carries an expandable balloon 388 and that is adapted to be positioned within a second location for stent implantation while proximal end portion 384 extends externally from the patient with a coupler 385 adapted to couple to a pressurizable source of inflation fluid, such as an indeflator filled with radiopaque contrast inflation fluid. Distal overlapping stent 390 is loaded onto balloon 388 with an overlapping end portion 392 located proximally of the rest of the stent 398 that includes the stent body and the opposite, proximal stent end portion. The stent assembly system depicted in FIG. 17B also includes marker bands 391,393,395.

In the embodiments shown in FIGS. 17A and B, the proximal overlapping stent 360 and the distal overlapping stent 390 are adapted to be overlapped with each other in a similar manner described above by reference to FIGS. 15A-16C as follows. In one mode, proximal overlapping stent 360 is implanted in an expanded configuration at the first implant location along a vessel wall such that distal overlapping end portion 362 is located distally within the vessel relative to the remaining portion 368 of stent 360. Thereafter, distal overlapping stent 390 is implanted at the second implant location such that proximal overlapping end portion 392 overlaps with distal overlapping end portion 362. This provides the relatively continuous diameter D along the stented region, including at the area of overlap between end portions 362,392.

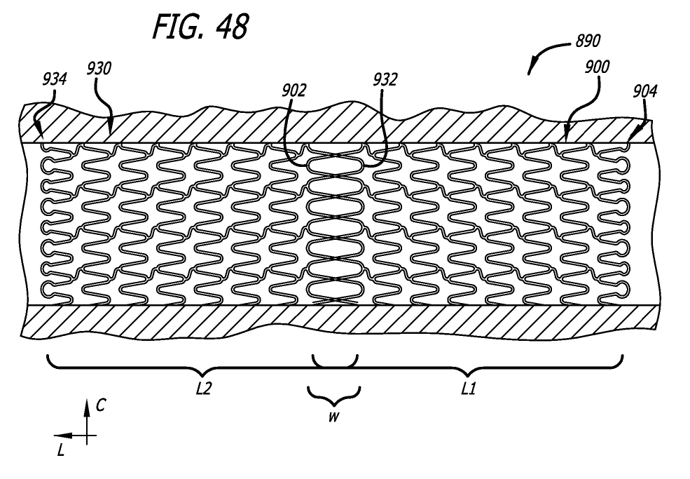

An exemplary embodiment depicted in FIG. 48, depicts overlapping stent assembly 890 implanted within a vessel. Stent 930 comprises a first end 934 and a second end 932. Stent 900 comprises a first end 902 and a second end 904. The first end 902 of stent 900 and the second end 932 of stent 930 overlap by a width w.

It is to be appreciated that the detailed arrangement and modes of use are variations to be contemplated by one of ordinary skill based upon other modes. For example, the distal stent may be placed first, followed by the proximal overlapping stent. In fact, in this particular arrangement, one stent is not required to be crossed through the lumen of the other prior to overlapping implantation. It is also to be appreciated that these two delivery systems may be packaged separately, or together as a kit. Either type, proximal or distal overlapping stent, can be used in combination with another type of stent, e.g. a conventional stent, or both may be used together as just described. By providing both on a physician's shelf, he is able to choose the type he requires to overlap with a first implanted stent, such as when only one end needs further stenting, e.g. in response to a dissection. It is also contemplated that whereas long stents may become more prevalent in the DES age, they do not meet all requirements in wide clinical practice. In one regard, their length at times limits their ability to track to and cross certain lesions requiring stenting. Two overlapping stents of shorter length will perform better than a single long stent in certain circumstances. In addition, any stent implantation carries the risk of an edge dissection following high pressure dilatation. Even long stents may benefit by the provision of an overlapping stent with reduced profile end for the overlap zone. In any event, a stent is provided with lower strut thickness profile at least at one end for improved overlap characteristics with another stent, and these various aspects are thus considered broadly beneficial despite the particular implementation chosen (which may itself provide further substantial benefit for certain circumstances).

It is further contemplated that the modified structure at the overlapping stent ends provides for a locally modified radiopacity resulting from a different density of the metal pattern there. This change has been observed to be fluoroscopically visible for certain particular arrangements, and aids a treating physician in implanting the stents with repeatable, controlled degree and location of overlap. In embodiments depicted in FIGS. 17A and B, marker bands 361,363,365 and 391,393,395 are included for this purpose.

According to the foregoing description, various different types of modifications to the ends of stents have been described in various levels of detail. For the purpose of further illustration, the following provides further clarity to certain such aspects, as well as further detail of certain further embodiments to illustrate the broad scope of the intended aspects of the disclosed stent assembly system.

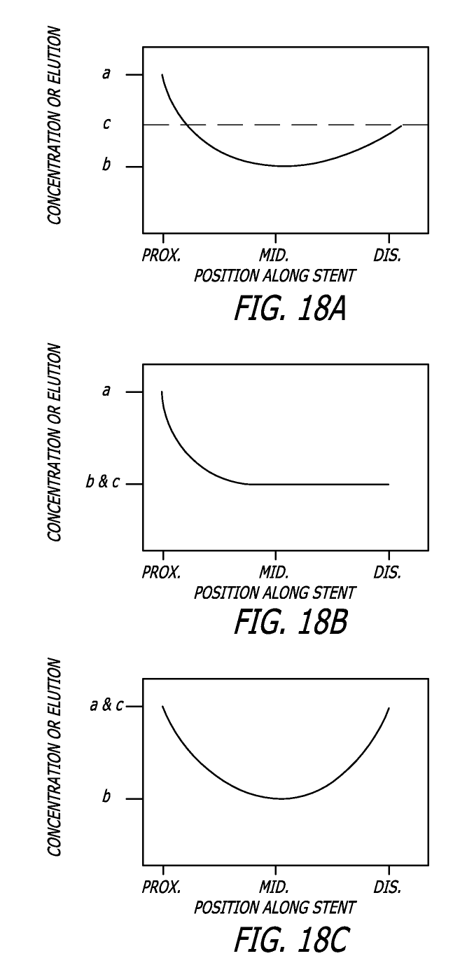

For example, FIGS. 18A-C variously show certain schematic graphs illustrating the effects of certain embodiments related to locally customizing drug delivery with a varied gradient along a stent's length, and in particular relative to the stent's ends. As noted above, along a stent's length, drug elution concentrates at the center body regions where all vessel portions are surrounded by drug-carrying scaffolding. The impact of the drug elution on restenosis at the stent's ends is less, and in particular at the proximal end. Vaso vasorem and other active and passive transport mechanisms may transport drug in a proximal to distal fashion, and thus the proximal margin may see less prolonged drug exposure whereas the central body or distal margin regions of the stent the downstream “wash” may provide higher drug concentrations there. In any event, it is axiomatic from clinical trial data according to varied DES products and related stent chasses and drugs used that the proximal edge experiences more restenosis than other regions. Accordingly, higher concentration of drug dosing at the proximal end versus other region of the stent is indicated to further increase drug therapy there, overcoming the particularly higher obstacles in the region, and thus reduce restenosis.

Various drug gradients along the stent length are thus contemplated. In one regard, as shown in FIG. 18A, the proximal end portion of a stent is provided with a drug elution profile designated as “a”, the mid-body portion exhibits a profile designated as “b” that is less than “a”, and finally the distal end portion exhibits a drug elution profile “c” that is yet again higher than “b” in the body portion but not as high as “a” at the proximal end portion. This particular gradient scheme maps against known clinical data for restenotic activity along the lengths of stented segments using certain leading DES products, more specifically particular focal frequency at the proximal end, followed by reduced but nevertheless still heightened frequency at the distal end portion, and then followed by lower incidence along the stent body. These differences are considered on a “density” basis per unit area of vessel. For example, an 18 mm stent may have a 3.2% restenosis “in stent” between the stent ends, but the rate is still lower, as is the density of drug need, per unit area when compared with a 2% rate at for example the distal end that only considers the restenosis over a 5 mm length of vessel.

It is contemplated however that other gradients may be suitable and beneficial compared to conventional approaches that treat designs and drug elution the same along the whole stent length. For example, FIG. 18B illustrates an elution profile with a heightened profile equal to “a” at the proximal end portion, whereas the mid-body and distal ends at “b” and “c” are equal in their elution profiles. This is indicative, for example, where designs are chosen to enhance drug elution at the proximal end that are not advantageous, or otherwise disadvantageous, at the distal end. As discussed above for example, end crowns with enlarged bulbous shapes may provide highly beneficial density and amount of drug elution at the stent edge, but due to an overlapping arrangement in a rolled down configuration may increase profile (a result that may be acceptable on the proximal stent edge, but not on the distal stent edge).

In yet another gradient example for further illustration shown in FIG. 18C, the proximal end distal end portion may exhibit equally elevated elution profiles “a” and “c”, whereas the mid-body portion has a relatively lower profile at “b”. This may be illustrative for example of incorporated stent scaffolding designs for increased drug elution at the ends that otherwise would not be well suited along the entire length of the stent body. For example, arrangements noted above that “close the gaps” between end crowns may create a harmful design everywhere on the stent where certain gaps between crowns and struts are required to provide for suitable vessel side branch patency and flow. By providing such only localized along short lengths at the ends, the benefits may be well provided without the accompanying risks for side-branch closure as the feature is highly localized and can be accurately placed in a vessel.

It is to be appreciated that these gradients just described, while each being highly beneficial according to certain particular embodiments and circumstances of use, are illustrative and may be modified to suit a particular need without departing from the broad scope of certain aspects herein described. For example, these graphs are illustrative and not to scale, and the various ratios between data points described may be modified. Furthermore, while the gradients are shown as curvilinear rates of change along the length, they may be stepped or otherwise modified according to one of ordinary skill.

According to other additional considerations, shear at the ends of stents is also believed to play a role in restenosis there. Thus, providing anti-platelet aggregation or anti-thrombin agents (e.g. clopidogrel or PLAVIX™, Heparin, IIb/IIIa inhibitors, etc.) in particular at the stent ends may provide substantial value. While such drug agent may be provided along the mid-body portions of the stent as well, certain embodiments herein contemplated provide them only at the ends, and in further embodiments only at one end, and in particular at the proximal end. Moreover, inflammation is also considered a culprit in the edge effect of restenosis. Thus, an anti-inflammatory compound such as dexamethasone, in particular therapeutic modes at the stent ends, is also contemplated.

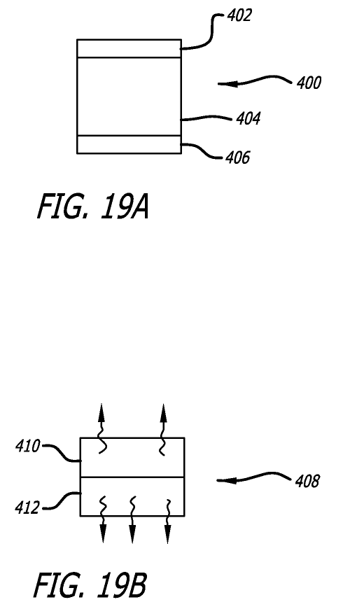

According to a further embodiment, a stent is coated with multiple compounds, at least one on a lumen side as a platelet aggregation inhibitor or thrombin inhibitor (e.g. clopidogrel or heparin), and on the wall side of the stent strut is an anti-restenosis agent (e.g. paclitaxel, sirolimus, erythromycin, exochelin, estradiol, everolimus, tacrolimus, desaspartate angiotensin I (DAA-I), sialokinin, nitric oxide or nitric oxide donors or producers, or prodrugs or analogs or derivatives, or blends or combinations, thereof). Such may be coated in this varied manner an opposite surface coatings 402,406 of the cross-section of the stent strut 404 as shown for stent 400 in FIG. 19A, or may otherwise elute in this varied manner from two portions 410,412 of the stent strut 408 as shown schematically in FIG. 19B.

The present disclosure in one regard provides unique local structures along the ends of the stent where tissue-stent interface factors provide unique concerns, as shown and described variously with respect to Figures previously introduced above. Further embodiments are herein provided below in order to provide additional examples to illustrate the broad aspects contemplated herein, as well as particular implementations that are considered of particular benefit for certain circumstances.

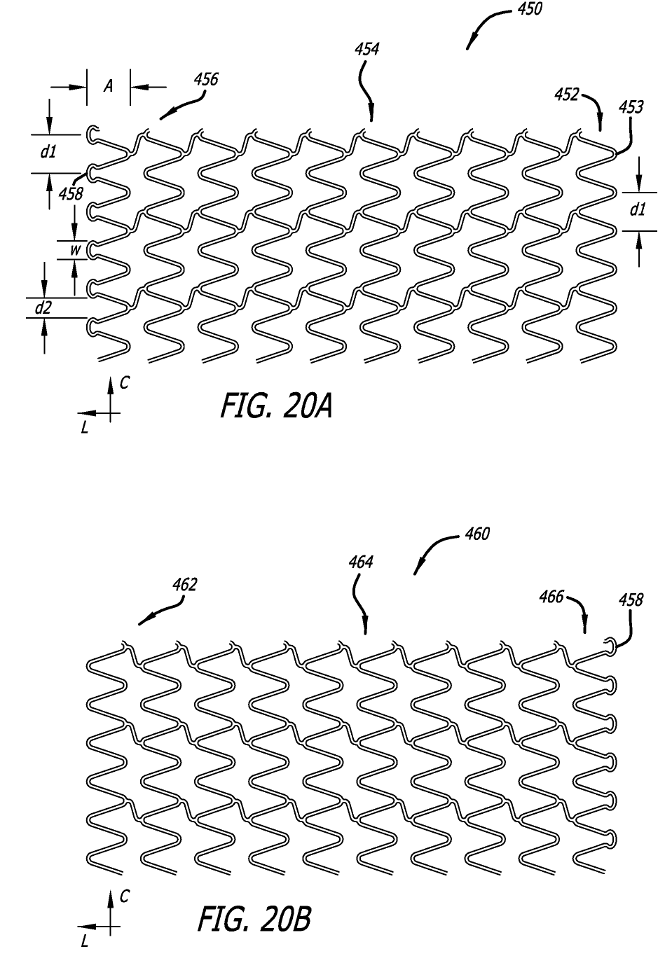

According to the embodiment shown in FIG. 20A, a stent 450 includes a first end portion 452, body portion 454, and second end portion 456. First end portion 452 includes an array of end crowns 453, and second end portion 456 includes an array of end crowns 458 that have unique features relative to first end portion 452 and the body portion 454 as follows. In general, second end portion 456 is shown on the left side of the Figure and corresponds to the proximal or upstream end of the stent 450 as it is to be loaded onto a balloon or other delivery mode and as intended to be implanted at a lesion site. The stent 450 includes a plurality of stent strut scaffolding segments along a linear array relative to the longitudinal axis L and that include stent strut scaffolding segments at the two end portions 452,456. The strut segment shown at this end 456 has a plurality of undulations between body crowns and end crowns 458, in many aspects similar to other strut segments along the stent length.

However, the end crowns 458 at this proximal stent end portion 456 are modified to include enlarged, partially oval structures at the crown 456. Each of these end structures has a radius of curvature at the actual peak which is larger than the radius of curvature elsewhere on the stent segments. Because these ends mark the area of dramatic tissue-stent transition interface, the larger radius structures are less penetrating, and are adapted to provide a more gentle structure against the tissue, especially when “over-expanded” (e.g. 5-10% over the adjacent reference luminal or vessel diameter). Moreover, these larger radius structures provide more surface area at the end of the stent for drug delivery in DES embodiments, and shorten the gap between crowns at the stent end, thereby increasing the density of the pattern for drug delivery at the stent end 456 as follows. The period or distance of one stent cycle from end crown to end crown on the proximal end portion 456 is indicated as d1, and in the particular embodiment shown is similar to the pattern with respect to period d1 and amplitude A as the rest of the stent body 454 and opposite end portion 452. However, due to the width w of the crown enlargements, the distance between adjacent sides of end crown enlargements is indicated as d2, clearly less than the distance d1 defined by the cycle period. These crowns 458 thus “close the gap”, or at least confine them to shorter distance with more drug delivery scaffolding there.

FIG. 20B shows another stent 460 that includes a first end portion 462, body portion 464, and second end portion 466. Second end portion 456 includes an array of end crowns 458 that have unique features relative to first end portion 462 and the body portion 464 as follows. In general this stent 460 is the same stent as FIG. 20A, provided that the end crown structures of the first and second ends are interchanged, such that the unique end crown structure 458 is located on the downstream distal end 466 indicated at the right side of the page. This figure is provided in part to illustrate that the various end crown features herein shown and described may be located on either end of the stent with respect to proximal or distal orientation within a vessel or by reference to a delivery catheter system to which such stents will be coupled. Accordingly, the particular orientation on the right or left side of the page for the figures is contemplated to be illustrative, and may be either proximal or distal. Moreover, such end structures generally may be provided on both ends rather than only one end. However, as elsewhere herein indicated, particular benefit may be gained from providing certain of these embodiments on only one end of the stent, and in particular the proximal end.

For example, it is noted that this increased mass at the end crowns 458 indicated in FIGS. 20A and B may provide for increased crossing profile on the respective end carrying the structures. This design concern is significantly more crucial on the distal end of the stent which encounters the most challenging crossing requirements. Accordingly, though each embodiment, and their combination, is considered within the scope of the invention, the embodiment providing such locally unique, larger radius end crown structure is considered in particular beneficial when provided on the proximal end of the stent alone.

Various modifications of the broad aspect of the stent delivery system exemplified in the embodiment of FIG. 20A-B and previously above with respect to uniquely arranged scaffolding (and thus drug delivery vehicle) structures at stent end portions may be made without departing from the scope of the stent system, as further illustrated in the following figures.

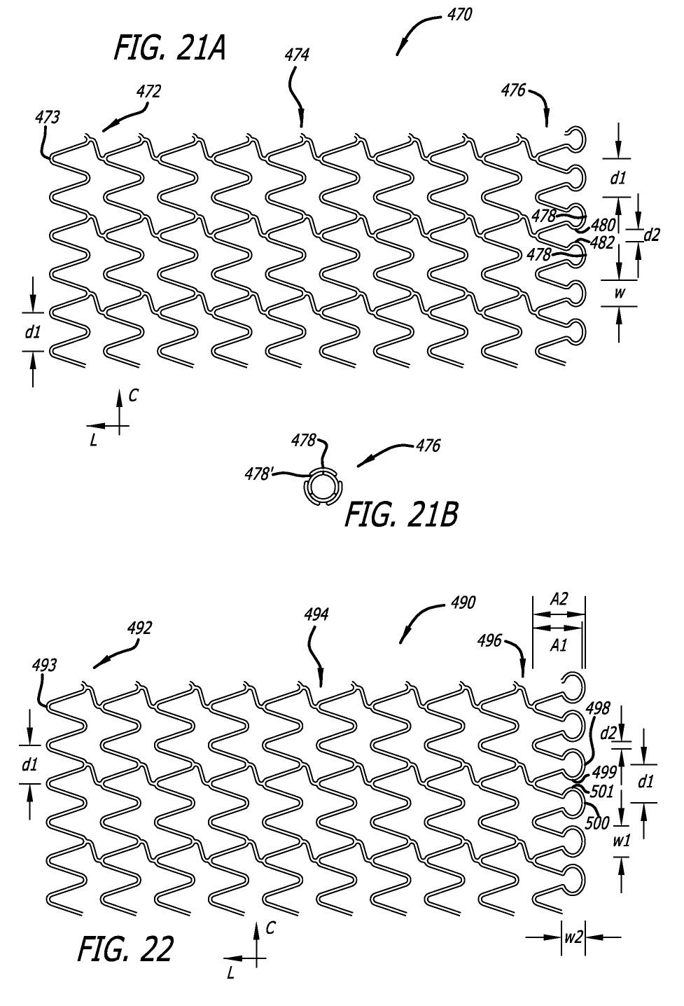

For example, FIG. 21A shows a stent 470 with a first end portion 472, body portion 474, and second end portion 476 that has end crowns 478 that are of unique design with respect to the rest of the stent including along body portion 474 and the end crown structures 473 at the opposite end 472. This embodiment is similar to that shown in FIG. 20A-B, except that the end crowns 478 are still further enlarged bulbous structures relative to the converging struts that form the base of these end crowns. These end crowns 478 have a width w such that the inter-crown distance between facing sides 480,482 of adjacent end crowns is only d2. This distance is a still smaller gap between crowns than the unique end crowns 458 of the FIG. 20A-B embodiments, despite having a similar periodic distance d1 for the full cycle of the undulations at the end portion 476. This further impacts the amount and density of drug elution into tissue at the stent end 476. Again, though shown on the right side of the Figure, and though possible to be provided on either or both of ends 472,476 of the stent 470, the particular benefit from providing these structures 478 on only the proximal end crowns of the stent 470 is further increased with this embodiment. The overlapping rolled-down arrangement shown in FIG. 21B might provide still further increase to profile which may be acceptable at the proximal shoulder but much more deleterious to crossing performance if provided on the distal end of the stent 470.

For further illustration, FIG. 22 shows a stent 490 with a first end portion 492 with a first configuration of end crowns 493, body portion 494, and second end portion 496 with a second configuration of end crowns 498 that are unique relative to the rest of the stent including the body portion 494 and the opposite end crowns 493 on first end portion 492. This embodiment illustrates a still further progression of these locally unique proximal end crown structures with still larger curvature bulbs at these crowns, and to the extent that the inter-crown distance is less than the distance between opposite lateral sides within the enlargements themselves. More specifically, end crowns 498 have a width w1 relative to the circumferential plane such that facing sides 499,500 of adjacent end crowns 498,500 provide a separation distance of d2, which is again smaller than the periodic distance d1 of the undulating pattern at the end portion 496, and still smaller than the separation distance d2 of the FIG. 21A embodiment.

It is further indicated in FIG. 22 that end crowns 498 also have a larger width w1 relative to the longitudinal axis than the similar dimension of the end crown embodiment in FIG. 21A. This widened dimension provides additional benefit as follows. As indicated by dashed converging reference lines at the bottom right-hand side of end portion 476 in FIG. 21A, the end crowns 478 of that embodiment terminate at a stent end that is essentially where the end crowns would have otherwise terminated if the bulbous features were not provided and their configuration were similar to that for crowns 473 on the other end of stent 470. However, as shown by reference to similar converging dashed lines in FIG. 22, the larger dimension w1 of the end crowns 498 in that embodiment lengthens the terminal end of the stent 490 slightly beyond where the converging end struts would have crossed as a conventional elbow design. This increases the amplitude uniquely at the end portion 496 segment from A1 to A2, and provides two functional differences. In one regard, the benefits of support scaffolding and drug delivery are extended into the “margin” area that might be injured during balloon expansion or otherwise post implantation. In another regard, the shape provides a different radius of curvature which may impact tissue erosion and inflammation reduction in the margin area.

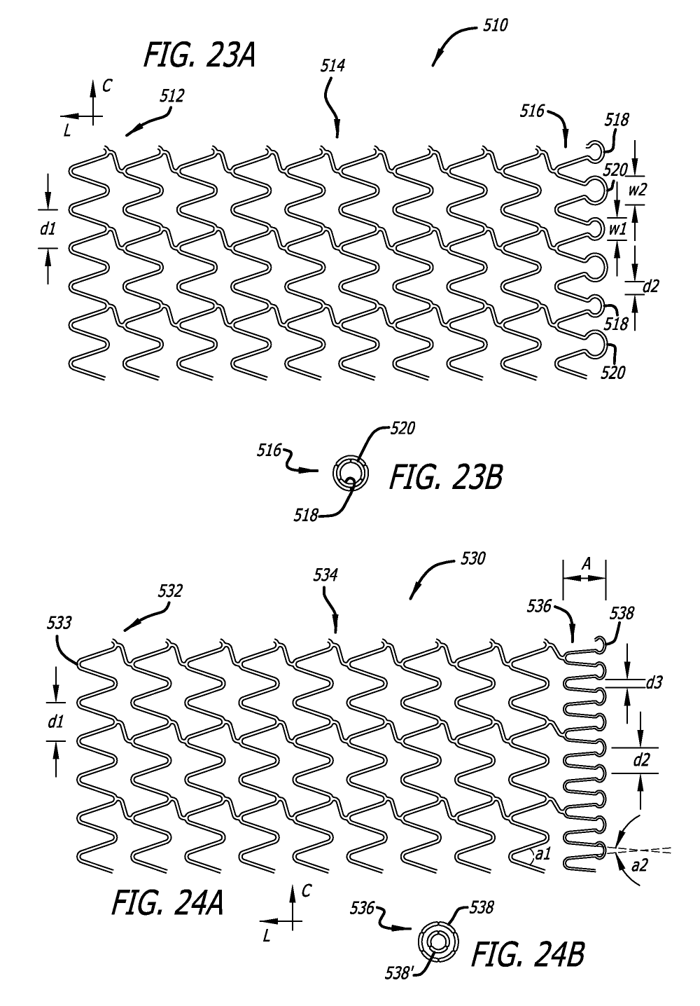

The unique local structures or enlargements may be further modified, and in fact multiple such structures may be incorporated at a single stent end, as shown in FIGS. 23A-B. FIG. 23A shows a stent 510 with a first end portion 512, a body portion 514, and a second end portion 516 that includes an array of two alternating types of end crowns 518,520 having varied respective widths w1, w2 that provide certain benefits as follows. Adjacent end crowns 518,520 are separated by a distance d2 that is smaller than the distance d1 of separation between other crowns elsewhere along the stent. As shown in FIG. 23B, and generally applicable to the prior figures, the transverse cross-sectioned view across this uniquely designed end 516 in the collapsed configuration requires that the end crowns 518, 520 overlap. When so overlapped over a balloon skin, such may provide for increased profile (versus designs where only one strut width is provided over the balloon, the present embodiments require two struts additive to the profile). According to the FIG. 23A-B embodiment, end crowns 518 intended to be located on the internal aspect of the overlapping arrangement are provided with smaller diameter crown enlargements (w1) than the alternate end crowns 520 which are intended to rest over those first smaller end crowns 518. This maximizes the ability to provide a compact collapsed configuration notwithstanding the overlapping, thus minimizing the profile impact.

It is to be appreciated that the previous embodiments described immediately above generally differ with respect to the size and shape of particular end crowns located at one end of the stent, whereas the other stent features are similar between the embodiments, including at the end portion that includes the unique end crown structures. However, it is contemplated that in addition to size and shape of end crowns, various other parameters may be modified to provide for the unique local structure and related benefits at an end portion of the stent.

For example, as shown in FIG. 24A, a stent 530 includes a first end portion 532 with an array of one type of end crowns 533, a body portion 534, and a second end portion 536. End portion 536 has a locally unique stent scaffolding pattern that includes a denser packed design to the stent segment there, but with thinner strut thickness than provided elsewhere on the stent. More specifically, the end portion 536 has an increased frequency of undulations along the circumference C, and thus provides a 9 crown design there versus a six crown design elsewhere along the stent including body portion 534 and opposite end portion 532. The unique frequency of the crown pattern results in a periodic crown-to-crown distance of d2 that is less than the full periodic crown-to-crown distance d1 elsewhere along the stent 530. Furthermore, by also providing bulb enlargements at the end crowns 538, the inter-crown distance at end portion 536 is further reduced to d3. According to the particular illustrative embodiment shown, the amplitude A of the undulating stent scaffolding segment that comprises end portion 536 is similar to that along other portions of stent 530, though may also be modified to meet a particular need.

This arrangement according to the embodiment in FIG. 24A provides for more surface area for drug delivery, while beneficially avoiding an increase to the overall stiffness of the end region due to the decreased stent scaffolding thickness there. In fact, certain particular configurations and combinations of dimensions and materials may provide a more flexible end portion 536 than the rest of the stent 530 despite the denser pattern. Increased density of drug delivery pattern into the lumenal wall is a benefit, especially when provided in combination with constant or improved stiffness transition at the stent end, and further including the beneficially enlarged radius at the end crowns to minimize trauma.

Further results of modifying the periodic frequency include modified angles of the respective struts that connect the crowns, as shown by comparing angle a1 versus a2 indicated in FIG. 24A. Such differing angles may also impact stiffness according to basic mechanical principles known to one of ordinary skill. As another result, 9 end crowns must now be given adequate real estate in a rolled down collapsed configuration for delivery, versus 6 crowns elsewhere on the stent. While this may be accomplished in a variety of ways, including a variety of overlapping crown configurations, one particular arrangement is shown in FIG. 24B wherein 3 certain ones of the 9 end crowns 538, designated as end crowns 538′, are given the inner radius real estate of the overlapping configuration, and the other 6 of the end crowns are arranged circumferentially thereover in the outer radius aspect of the collapsed end portion 536. For example, every third crown along the circumference may be given the inner radius position in this arrangement, whereas the intervening groups of 2 end crowns are arranged along the outer radius portion. A separation gap between these outer and inner regions is shown for illustration, though other arrangements may provide intimate contact in a compact manner.

It is to be appreciated that the end crowns such as crowns 538 just described by reference to FIG. 24A may be further modified in additional embodiments to provide more complex shapes to achieve the intended objects of the present aspects of the invention versus simply providing larger radius apices at the crowns.

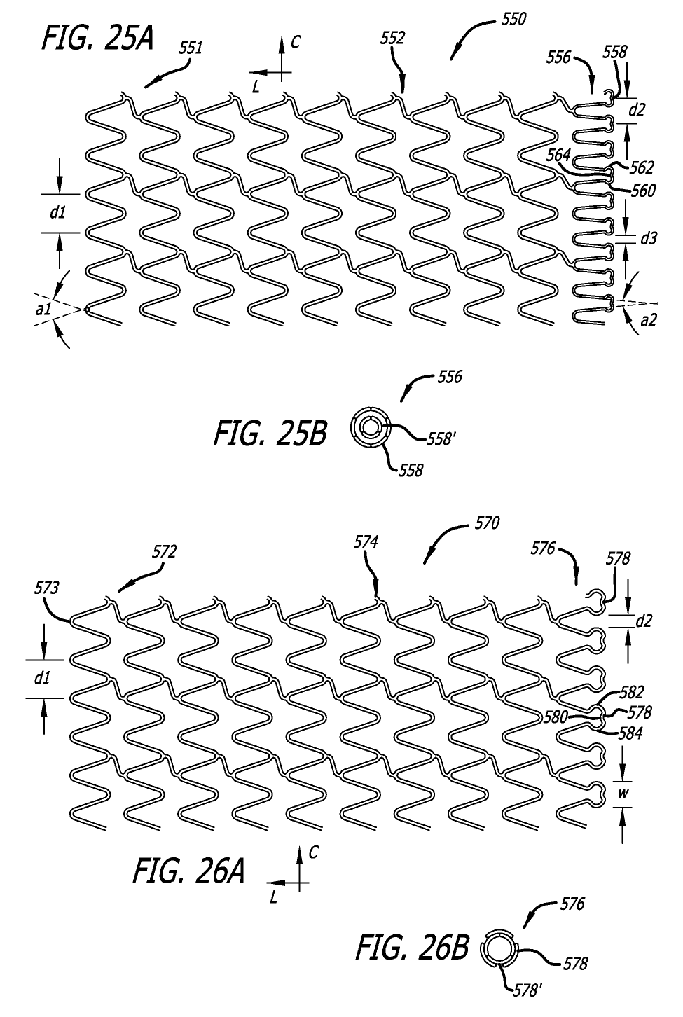

For example, as shown in one further embodiment in FIG. 25A (and still a further embodiment in FIG. 26A for further illustration), a stent 550 includes a first end portion 551, a body portion 552, and a second end portion 556 with an array of end crowns 558 that are unique structures relative to the rest of stent 550 including body portion 552 and opposite end portion 551. According to this particular embodiment, the array of enlargements or bulbs that form end crowns 558 are similar to that shown for FIG. 24A, including the increased periodic frequency of 9 crowns versus 6 along the rest of the stent, as well as the pitch angle a2 of struts between crowns that is varied from angle a1 along the other portions of the stent 550. However, more particular to this embodiment, the shapes at the end crowns 558 further include invaginations 560 that result in two mini-crown bulbs 562,564. These provide a yet further modified mechanical and drug delivery result there as a tissue implant along a luminal wall. As illustrated in FIG. 25B, such arrangement results in similar roll-down configuration with overlapping end crowns 558, 558′ for end portion 556 as that previously described for FIG. 24B.

It is further contemplated that other undulations or additional shapes may be incorporated to provide yet further surface area for increased drug delivery at those end crowns. Moreover, similar general structures may result in varied biomechanical and drug delivery results when the respective scale is modified.

For example, FIG. 26A shows a stent 570 with a first end portion 572 that includes an array of end crowns 573 spaced by a distance d1, a body portion 574, and a second end portion 576 having an array of end crowns 578 that are uniquely configured versus the rest of stent 570 including body portion 574 and opposite end portion 572. The various features related to FIG. 26A should be apparent to one of ordinary skill based upon the figure alone and in context of other disclosure provided hereunder, but are further described for illustration as follows. In this particular embodiment, end crowns 578 are of similar shape as those just described for end crowns 558 in FIG. 25A, and include invaginations 580 resulting in two mini-crown bulbs 582,584. These end crowns 578 have a width w such that the inter-crown distance d2 is less than the inter-crown distance between end crowns 573 on the opposite end portion 572 of stent 570.

However, differences in the embodiment of FIG. 25A when compared to the embodiment shown in FIG. 25A are as follows. This end portion 576 has a similar periodic frequency of undulations as the rest of the stent, a 6 crown design for all segments, and thus a full periodic crown-to-crown cycle at end portion 576 is a similar distance d1 as the rest of the stent. The shaped end crowns 578 are larger than those of the FIG. 25A embodiment. As a result, the effects of the invaginated shape may be different in the context of a similar vessel wall. For example, the radius of curvature of the mini-crown bulbs 582,584 are larger than those for similar structures 562,564, and such difference may have a different impact on tissue erosion or trauma. As also elsewhere described, the resulting pattern, though based on similar shapes, is different over a given circumference C, and thus drug delivery and mechanical scaffolding effects may differ. As further shown in FIG. 26B that illustrates end portion 576 rolled down with inner crowns 578′ surrounded by outer crowns 578, the impact of modifying the number of crowns at the end portion affects the respective overlapping roll-down configurations as well.

As illustrated in the immediately preceding embodiments, and others of the present embodiments, such locally unique stent strut structures at the end crowns allow for more drug, or higher density of drug over a give lumenal circumference, to be delivered at this region notwithstanding an even dose coating modality along the length of the stent and including these unique end structures. Accordingly, where more drug is desired at margins around the stent, for example to further retard restenosis at segments upstream from the proximal end (or downstream from the distal end), such may be accomplished with the locally modified stent scaffolding, and the coating or drug loading aspects need not be modified. This ability to provide variable drug delivery along the stent length, in particular at a stent end, and still more particularly at the proximal end, while allowing a constant coating modality along the stent, has significant manufacturing benefit. Else, without the unique structures herein provided to achieve this objective, multiple coating treatments must be done across very small dimensions, a proposition that is highly difficult to execute and extremely complex to control in any significant manufacturing scale.

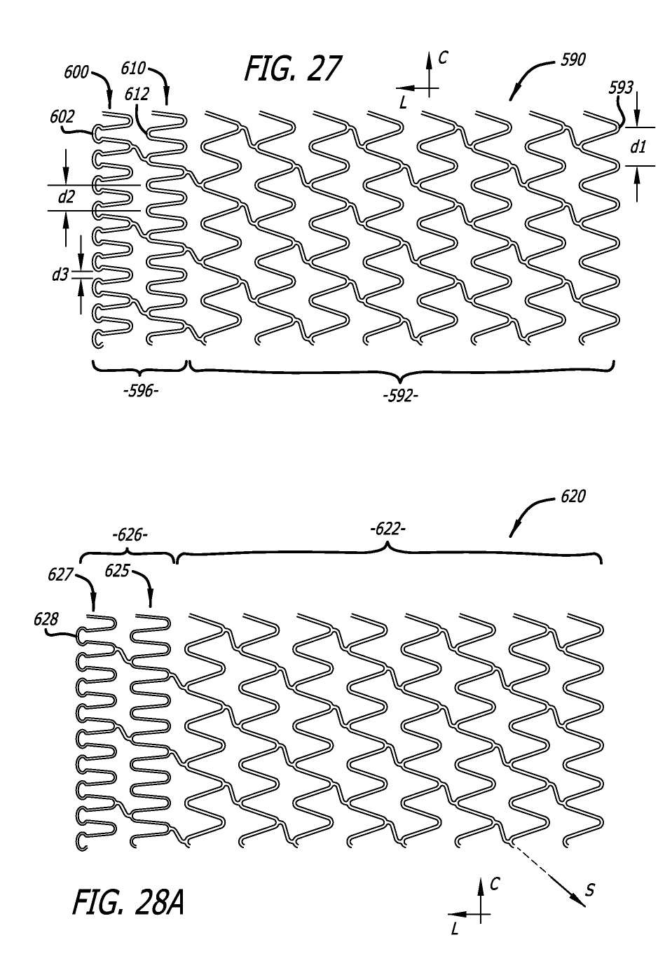

In still further embodiments, further aspects of the invention contemplate that the unique local structure or structures may be provided along a stent end portion that includes more than one stent scaffolding segment. For example, the embodiments shown variously in FIGS. 27-30B provide unique local structures on the end segments on the outer most periphery of the stent, as well as the next adjacent segment. According to these embodiments, a more thorough impact on the overall function at the corresponding end portion is achieved. This may provide in some instances more gradual transition from vessel to the bulk of the stent body than is provided by the previous embodiments above that treat only the very end segment with a unique structure. In addition, still further increased drug dosage may be provided over a larger region of the end portion where more trauma during the motion of the in-vivo heart cycle may be experienced.

More specifically, FIG. 27 shows a stent 590 with one end portion 596 that includes both a terminal end segment 600 and an adjacent end segment 610 having unique respective structures versus each other and the rest of the stent 592. More specifically, segment 600 comprises the terminal segment of stent 590 at end portion 596 and includes an overall strut/crown structure similar end portion 536 in the embodiment FIG. 24A, provided that in this particular embodiment the thicknesses of the scaffolding structure, e.g. struts, is similar to the rest of the stent and not of reduced thickness. In any event, the periodic crown-to-crown distance d2 between adjacent crowns 602 at terminal end segment 600 is less than the inter-crown distance d1 between adjacent faces of end crowns 593 at the opposite end of the stent 590 (and elsewhere on the stent beyond adjacent end segment 610). Circumferential enlargement bulbs provided on the end crowns 602 close the gap between these crowns to a reduced distance d3 for enhanced drug delivery at that end. Adjacent end segment 610 includes a very similar structure and pattern to terminal end segment 600, except for the omission of circumferential enlargements as bulbs at the respective crowns 612 along that segment. Accordingly, drug delivery is densified per unit area of surrounding tissue along the more compact scaffolding along end portion 596, whereas the bulbous crowns structures are provided only at end crowns 602 where tissue-device interface is a most pronounced concern.

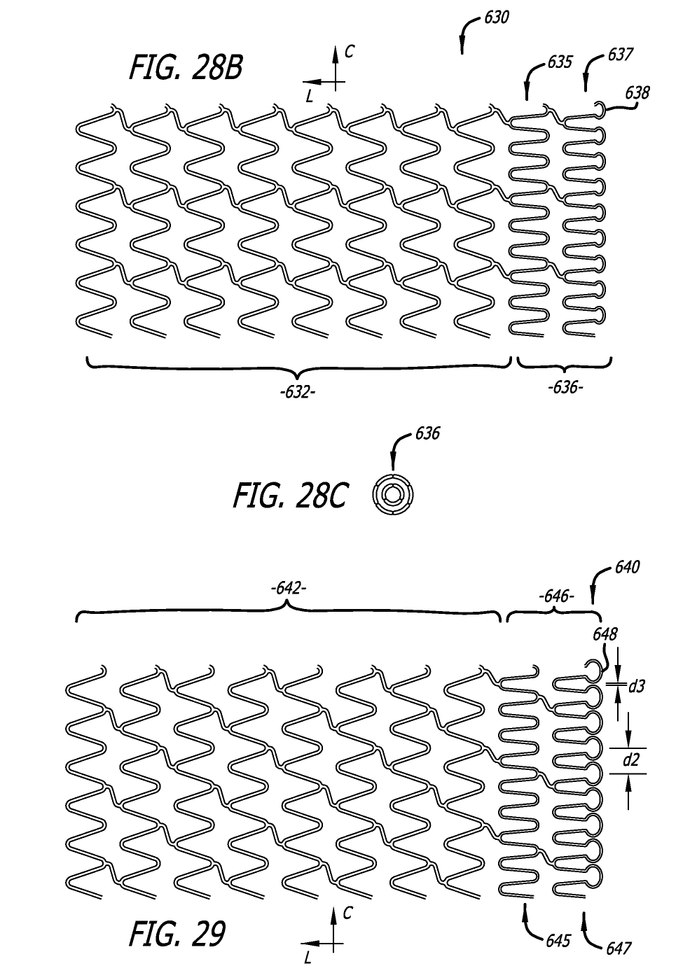

FIG. 28A shows a stent 620 that includes an end portion 626 with a terminal end segment 627 and an adjacent end segment 625 having unique structures relative to each other and the remaining portion 622 of stent 620. This end portion 626 is similar to end portion 596 shown in FIG. 27, including bulbous end crowns 628, provided that the thickness of the scaffolding in the region is reduced from the rest of the stent, similar to that described above by reference to end portion 536 in FIG. 24A. In this particular arrangement of densified pattern for two end segments of the scaffolding, such reduction in thickness may be of increased value, as the otherwise result may be a substantial stiffening of the stent at its end, generally a result to be beneficially avoided. As described by reference to other embodiments, this arrangement of FIG. 28 may be provided on either end of the stent, as illustrated by FIG. 28B that shows stent 630 with an end portion 636 on the opposite end of the remaining portion 632 of the stent 630, and including terminal end portion 637 with bulbous end crowns 638 and adjacent terminal end 635. An illustrative roll-down configuration applicable to either of FIGS. 28A or B is shown in FIG. 28C for example by reference to end portion 636.

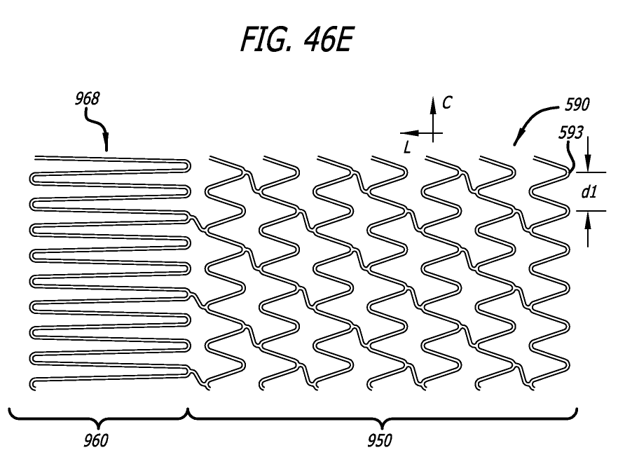

FIG. 46 depicts various configurations of stents having first end portions 952,954,956,958,960 with one or more elements 962,964,966,968, wherein the total longitudinal length of the first end portion does not exceed 4.0 mm. Each first end portion can include up to four elements wherein each element can independently having a longitudinal length from about 0.5 mm to about 3.0 mm. In certain embodiments, an individual element has a longitudinal length of 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm and 2.9 mm. In one embodiment depicted in FIG. 46A, first end portion 952 comprises four elements 962, each element 612 of equal longitudinal length. In another embodiment depicted in FIG. 46B, first end portion 954 comprises two elements 962,964, element 964 having a longitudinal length three times that of element 962. In another embodiment depicted in FIG. 46C, first end portion 956 comprises three elements 962,962,964, first element 966 having a longitudinal length twice times that of elements 962. In another embodiment depicted in FIG. 46D, first end portion 958 comprises two elements 966, each element 966 of equal length. In another embodiment depicted in FIG. 46E, first end portion 960 comprises one element 968.

It is to be appreciated that, as scaffolding patterns may be modified along a stents length to impact performance, the interconnects between adjacent segments along a length of a stent may also be modified, and also may impact stent performance. For example, as further illustrated in FIG. 28 by dashed reference axis S that is orthogonal to longitudinal axis L and transverse circumferential axis C, interconnects may be made in alternating fashion along a segment, and varied alternating fashion along an adjacent segment, to yield one or more orthogonal spines of interconnections. Such arrangement is shown for illustration in FIGS. 27 and 28, and vary from the interconnecting arrangement variously shown among FIGS. 20A-26B that are otherwise aligned as interconnect spines along the longitudinal axis of the respective stents. Such difference may impact certain performance results, including in certain circumstances yielding a different degree or pattern of “opening” experienced at the enclosed regions surrounded by stent scaffolding when the respective stent is put into a bend along a particular radius. This may impact trackability performance to get to and across a vessel blockage through certain degrees of tortuosity, and may also impact the density of drug delivery per unit area of surrounding tissue along the respective stent body. In other words, where such opening between stent segments is more pronounced, less drug is delivered to the tissue surrounding that area.

It is to be appreciated thus that many different types of interconnect patterns and structures are contemplated though they may not be particularly shown here, though certain particular patterns shown are considered in particular beneficial for certain optimal results. However, further modifications may be made by one of ordinary skill without departing from the intended scope of various aspects herein described, and in particular the beneficial features provided for end portions of stents are not intended to be limited in all cases to a particular body scaffolding or interconnect arrangement, though such may be of particular further benefit.

The particular embodiment shown in FIG. 29 further illustrates the extent to which end crowns may be enlarged to provide for enhanced drug delivery for DES applications according to various aspects of the present invention. More specifically, FIG. 29 shows a stent 640 that includes an end portion 646 with terminal end segment 647 and adjacent end segment 645 that include structures that are unique relative to each other and the remaining portion 642 of stent 640. The general pattern of undulating scaffolding for this embodiment is similar to that shown in FIG. 28B with similar periodic distance of undulations equal to d2, provided however that the bulbous shapes for the end crowns 648 are yet further enlarged from prior embodiments. In fact, in the expanded configuration shown, the enlarged crowns 648 nearly touch in this expanded, implanted condition with a substantially reduced distance d3. This illustrates a further progression of providing continuous drug delivery along the circumference of a stent end while maintaining a certain desired degree of flexibility as well as reducing potential trauma at tissue-device interface surfaces at the end crowns.

It should be noted that a further aspect of the stent assembly system provides structures that may not be generally manufactured using standard stent manufacturing techniques, wherein stents are generally cut from tubes or rings at sizes that closely approximate the desired collapsed configuration for the stent. Instead, according to these embodiments that require overlapping end crowns in the collapsed condition, the stents are generally cut for example from larger tubes that more closely approximate the expanded condition for the stent. In the present embodiment of FIG. 10, this stent would be cut for example from a tube sized very close if not equal to the expanded diameter for the stent as shown in the Figure. Thereafter, the stent would be crimped down to the collapsed configuration wherein the enlarged end crowns are overlapped. Subsequent balloon expansion merely returns the stent to the size at which it was first formed.

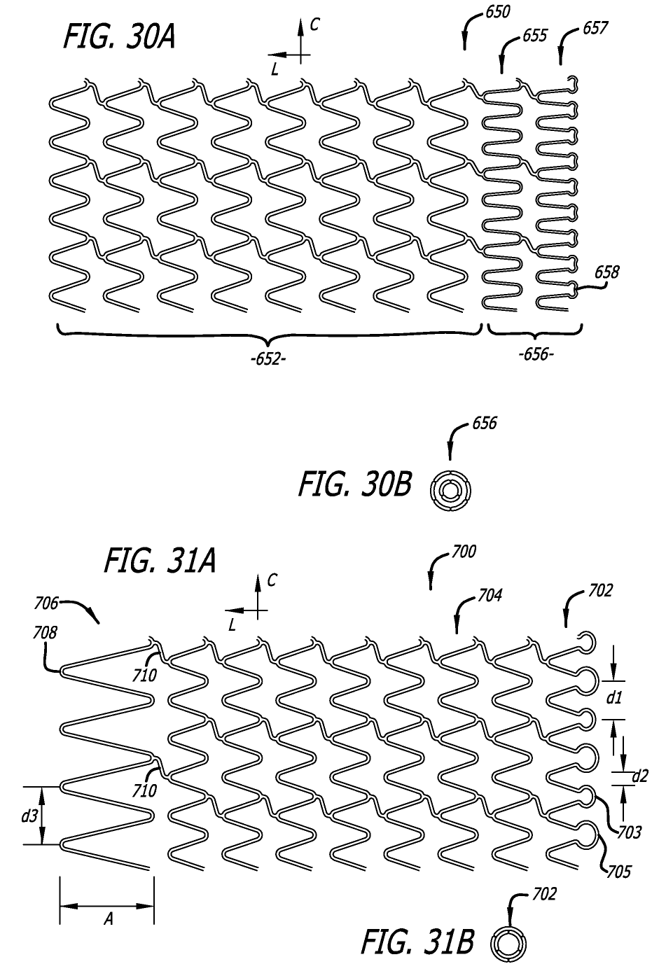

The various embodiments just described above may be variously combined or otherwise modified by one of ordinary skill in order to yield results consistent with the various objectives described. For example, FIG. 30A illustrates such combination wherein a stent 650 includes an end portion 656 with a terminal end segment 657 and an adjacent end segment 655 that have unique structures relative to each other as well as relative to the remaining portion 652 of stent 650. This arrangement is substantially similar to that shown and describe by reference to FIG. 28B, provided however that the end crowns 658 are configured with shapes similar to FIG. 25A. A roll-down configuration for end portion 656 is shown in FIG. 30B for further illustration.

For the purpose of providing further understanding of the overlapping stent embodiments described above by reference to FIGS. 15A-17B, further detailed embodiments are also provided as follows.

In one particular embodiment shown in FIG. 30A, a stent 700 includes a first end portion 702, a body portion 704, a second end portion 706 that includes a stent strut segment with a particular pattern of crowns 708 intended to overlap with another stent. More specifically, the overlapping end segment 706 has a single, undulating scaffold member that has fewer crowned undulations (n=4), each being expansive over a greater area than the other segments of the stent. The result is an inter-crown distance d3 that is greater than the periodic distance of undulations along the rest of the stent, and greater than the distances d1 and d2 between adjacent crowns 703,705 on the opposite end portion 702 of stent 700. In the particular embodiment shown, this amplitude A of the stent segment at overlapping end portion 706 spans a distance generally representing two adjacent segments of the stent body. Healthcare providers that intend to overlap stents typically target a repeatable portion to overlap, which in many cases is guided fluoroscopically and the objective is to achieve for example 5 mm of overlap between two adjacent 18 mm overlapping stents (or between about one-quarter and one-third of the stent lengths). Accordingly, the distance along which the unique overlapping segment may be provided may vary, though the arrangement shown is believed to be exemplary of a desired overlap.

In any case, according to the present overlapping embodiment much less stent material is provided in the intended overlapping area. In addition, the pitch of the resulting cycles of this segment is more acute to the longitudinal axis L of the stent and thus underlying vessel lumen. This resulting structure is believed to provide significantly improved hemodynamics when overlapped with another opposite end of a second stent, as will be further developed below. In addition, the transition in material density between the stent body and the overlapping end will provide substantial difference in radiopacity, allowing more precise placement of the overlapping stents in vivo.

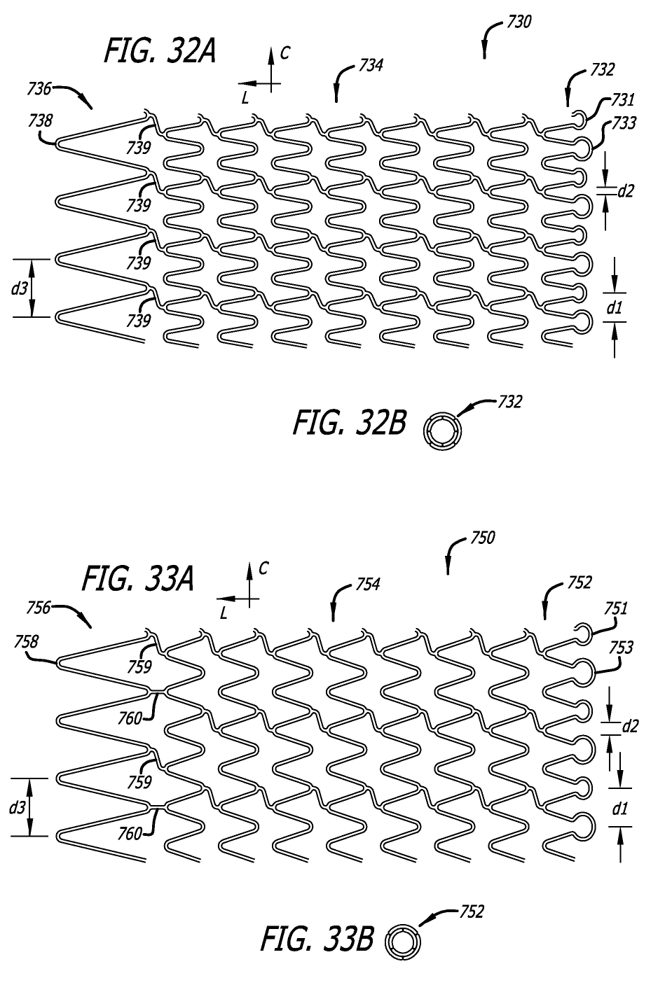

Further to the FIG. 30A-B embodiment, the opposite stent end 702 includes a unique local structure that differs from the overlapping end portion 706, and also differs from the segments within the body 704 of the stent 700 between the two ends 702,706. More specifically, for illustration this opposite end incorporates a similar unique local structure to that shown in FIG. 23A above. In the circumstance that this opposite end portion 702 is intended to be a proximal end for the stent 700 during use and implantation, the particular embodiment for the stent 700 in FIG. 31A is, in that case, illustrative of a stent intended to be the “proximal overlapping stent” in the overlapping arrangement. In other words, the overlapping end portion 706 represents the distal end of the stent as loaded onto the balloon or other delivery means, whereas the opposite end portion 702 represents the intended proximal end of the stent with the locally unique design intended to provide particular benefit at such proximal vessel-stent transition. However, as mentioned above, it may be reversed, though the benefits afforded by these particular local structures may be outweighed by sacrifices made to achieve them, such as for example distal crossing profile. Because the unique local structure provided at the overlapping end portion 706 of the embodiment of FIG. 31A relates to the number and spacing between crowns of the end strut segment, the junctions 710 between such overlapping end segment 706 and the different cycle of undulations for the adjacent segment may require particular attention in providing an overall device with optimal performance. Accordingly, further modifications and embodiments are variously shown among the following figures to provide further illustration of different junctions and relationships between overlapping ends and the adjacent stent body according to the invention. FIG. 32A illustrates one such modification having differences among features compared to FIG. 31A as would be apparent to one of ordinary skill, which are nevertheless further described for illustration as follows. FIG. 32A shows a stent 730 with a first end portion 732, body portion 734, and second end portion 736. First and second end portions 732,736 have unique local structures relative to each other and to the body portion 734 of stent 730. Body portion 734 has an 8 crown design with respect to each adjacent stent segment, whereas the body portion 704 in FIG. 31A has a 6 crown design for the segments. This 8 crown design carries through to end portion 732, and as a result certain alternate end crowns 731 there are slightly modified to smaller dimensions versus the alternate end crowns 703 in FIG. 31A in order to provide the requisite real estate along circumference C for all the end crowns along the end portion 702. Overlapping end portion 736 includes a similar 4 crown design as shown in FIG. 31A. However, according to the modified crown pattern of the adjacent segment of body portion 734, the difference between the crown patterns is an exact having of the periodic distance between crowns. As a result, a repeatable arrangement of similar shaped interconnects 739 is shown to connect the inward body facing crown portions of end portion 736 with the adjacent crowns. This differs from the FIG. 31A embodiment wherein the overlapping end portion crown pattern was a 4 crown design, and the adjacent segment had a 6 crown design. According to this ⅔ period ratio between segments, FIG. 33A shows an arrangement similar to FIG. 31A, except illustrating an interconnect arrangement at each inward facing crown at overlapping end portion and the adjacent segment. More specifically, FIG. 33A shows a stent 750 with a first end portion 752, a body portion 754, and a second end portion 756 adapted to provide an improved overlap coupling with a second stent. This stent 750 is similar to stent 700 in many substantial regards, including alternating end crowns 751,753 of different geometries and adapted for overlapping roll-down configuration as shown in FIG. 33B. However, in the present embodiment interconnects are provided at every crown undulation on the body side of overlapping end portion 756. More specifically, interconnects 759 are similarly arranged as interconnects 710 in FIG. 31A, and provide a shape adapted to bridge between the inward body-facing crown of the end portion 756 and the opposite facing crown of the adjacent body segment that are off-axis from each other relative to longitudinal axis L. However, as these are arranged at every other alternate body crown of end portion 756, the embodiment of FIG. 33A provides additional interconnects at the crowns otherwise not connected in FIG. 31A. These are aligned with the adjacent body crowns relative to the longitudinal axis L.

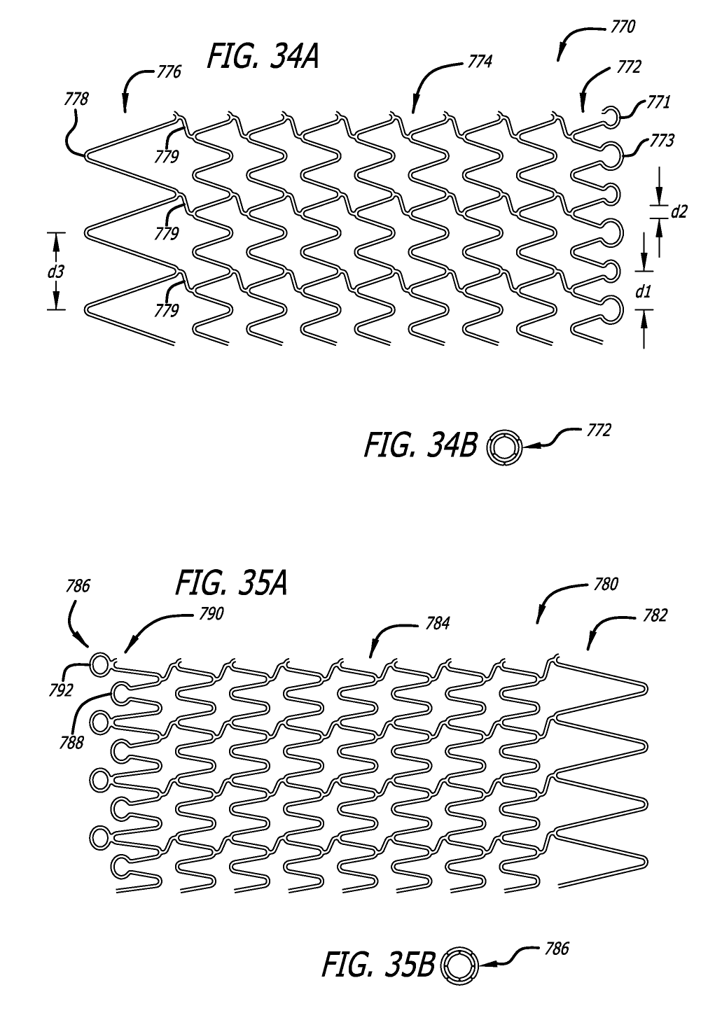

According to still a further modification, FIG. 34A shows a stent 770 with a first end portion 772, body portion 774, and second end portion 776 that is adapted to provide enhanced overlap coupling with a second stent. More specifically, overlapping end portion 776 includes an array of three overlapping crown structures 778, whereas the adjacent body structure is of a 6 crown design. This stent is similar in most substantial ways to the configuration shown in FIG. 32A, except for the three crown overlapping end portion 776 versus the four crown end portion 756 in the previous embodiment. This difference provides for a different interconnect arrangement according to the 3:6 crown ratio, again providing a half-periodic cycle and ability to provide a repeatable shaped interconnects 779 between segments. In addition, another result of the difference between the present embodiment and that of FIG. 33A is a wider gap d3 between end crowns 778 at the overlapping end portion 776. While these gaps were intended to be closed at stent end portions according to other embodiments where the end portions are intended to be at a tissue-device interface at the end of a stented segment, the more expansive gaps are well suited for certain different objectives of a stent end intended to overlap with a second stent. In this case, by overlapping with another stent also with three crowns at mating end portion, the resulting overlapped zone provides essentially a 6 crown design, and thus more continuity along the overall stented vessel segment. The gaps such as shown at d3 are, in that overlapping arrangement, surrounded by stent scaffolding according to the overlapping junction with the other second stent.

For purpose of providing further illustration of the many different arrangements and combinations of embodiments herein contemplated, FIG. 35A shows a particular further embodiment that provides a new structure at the opposite end portion opposite the overlapping end of an overlapping stent as follows.

FIG. 35A shows a stent 780 with a first end portion 782, a body portion 784, and a second end portion 786. First end portion 772 and body portion 774 provide similar stent scaffolding arrangement as previously described by reference to FIG. 32A at end portion 736 and body portion 734 according to a 4:8 crown ration design. However, in the present embodiment, the opposite end portion 786 includes a novel end crown structure. More specifically, an array of end crowns 790 alternate with an array of end crowns 788 each having a different configuration of bulbous ends. Whereas end crowns 788 are similar to those crowns 733 shown in FIG. 32A, the other alternating end crowns 790 instead form elbows as apexes to the converting struts there that terminate along the longitudinal axis generally aligned with the longitudinal termination of the end crowns 788. However, extending further from these elbows of end crowns 790 are enclosed circular bulbs 792. These extend the mechanical and drug elution characteristics of the stent beyond the typical end and into the margins where restenosis is known to occur. In addition, the circular bulbs 792 are beyond the bulbous end crowns 788, which removes certain interference considerations during roll-down, shown in end view in FIG. 35B.

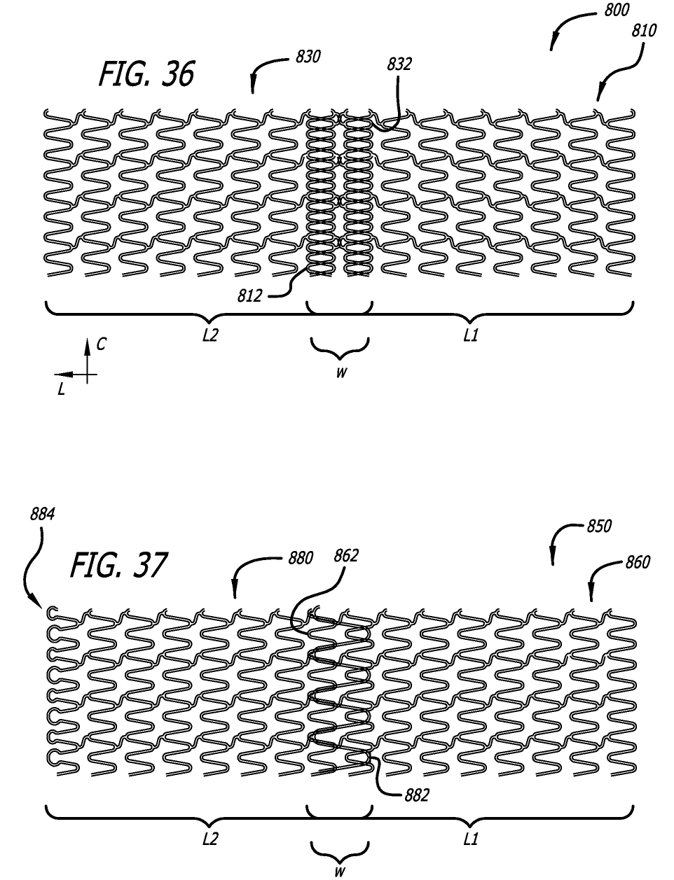

The various benefits of the overlapping stent aspects of the invention as illustrated above by reference to the various particular stent design embodiments is further illustrated below by reference to FIGS. 36-38 showing examples of their in vivo use for overlapping with adjacent stents as follows.

For an initial understanding, FIG. 36 shows an illustrative example of an overlapping stent system 800 with two overlapping stents 800,830 of conventional design. As is shown, by overlapping their lengths L1 and L2, an overlapping zone w results with a highly dense pattern of overlapping struts. While the particular orientations may vary on a case by case basis (it is difficult to control orientation of overlapping struts during percutaneous transluminal procedures under fluoroscopy), in the orientation shown each of two regions 812,832 of overlapping segments results in 16 locations of overlap, which together with 4 overlapping junctions provides 36 localized areas where struts cross. Moreover, the crossing undulations with opposite orientations provide 32 discrete cells along overlap zone w where the internal struts are raised from the vessel surface, resulting in sub-optimal hemodynamics and thrombogenic zones. Still further, it is further a consideration that, in the conventional DES setting, such doubling of material in the overlap area results in doubling of anti-restenosis drug dosing. In the case of many leading compounds for such indication, such may lead to cytotoxicity.

In contrast, FIG. 37 shows a similar view of another overlapping stent system 850, except including an overlapping stent 880 according to certain aspects of the present invention in overlapping arrangement with a conventional stent 860 that does not provide a unique overlapping region intended to mate with the present device. As is shown, the overlapping lengths L1,L2, respectively, result in an overlapping zone w with a less dense mesh of overlapping struts than previously shown in FIG. 36. Only 8 areas of strut overlap are shown on one overlapping segment 882, and 8 areas are shown where struts are substantially aligned across the other overlapped segment 862. It is believed that this result provides for much improved hemodynamics in the high blood flow, recanalized lumen, and hence reduced thrombogenicity. Moreover, the reduced density of stent material, in a DES embodiment, results in less drastic increase in drug dosing (or, conversely, more continuity along the stented region).

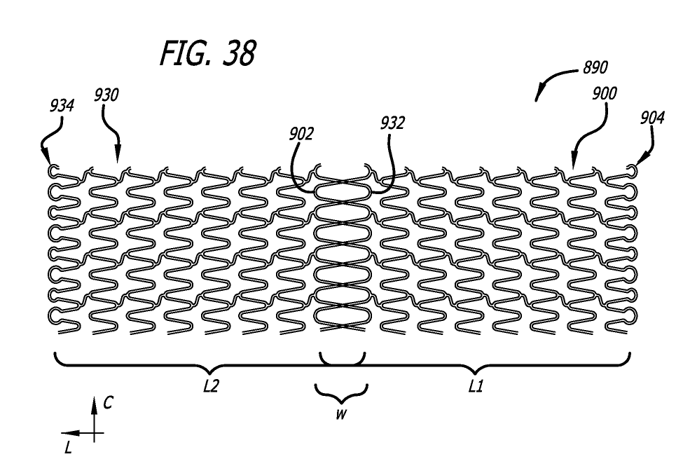

Still further, FIG. 38 shows an overlapping stent system 890 with two overlapping stents 900,930 according to certain aspects of the invention and are provided together in a combination assembly and method. Here, the overlapping lengths L1,L2 result in an overlap zone w with only 8 areas of strut overlap in the entire combination. Moreover, the resulting lattice structure across the entire stented region is more uniform, with more uniform structural support and drug delivery (to the extent such stents are adapted as DES devices).

It is to be further appreciated that such overlapping regions may benefit from reduced strut thickness, as well as incorporation for elution of various known compounds that may improve the hemodynamics, or at least thrombo-resistance, in the area. For example, hirudin, heparin, coumadin, clopidogrel, IIb/IIIa inhibitors, abciximab, or other anti-thrombin or platelet aggregation inhibitors may be incorporated into the entire overlapping stents, or in the region intended for overlapping, either alone or in combination with anti-restenosis drugs. Or, the thrombus affecting compounds may be adapted to elute from the lumenal side of the overlapping struts, whereas anti-restenosis compounds elute from the vessel wall side of the strut. Moreover, kits may be provided with proximal and distal overlapping stents with proper orientation for the intended overlapping arrangement for patient treatment. For further illustration of the various combinations of features and benefits contemplated, stents 900 and 930 are further shown to include end portions 904,934, respectively, that are opposite the overlap zone w and are consistent with certain beneficial embodiments for stent edges described elsewhere in this description.

Further modifications may be made to the foregoing embodiments without departing from the broad intended scope of the stent system. For example, additional unique local structures may be provided at stent ends, in particular proximal stent ends, in combination with or instead of the particular embodiments herein shown and described, which despite their particular benefits are also intended to be illustrative of broad aspects of the system. For example, one of the overlapping stent embodiments previously shown and described above also provide unique local structures on the end crowns opposite the overlapping end of the stent which provide enclosed rounded members intended to extend the “reach” of the stent to “in segment” tissue that is not otherwise being supported by the stent. This is in particular believed beneficial for drug delivery applications, and further in particular when locally provided specifically on the proximal end of the stent. Such may be further included on a stent not including an opposite overlap region, as further illustrates modifications that may be made without departing from the intended scope.

Various numbers are provided throughout the figures for the purpose of further illustration and are representative generally of stent embodiments cut to have expanded configurations shown as approximately 3.0 mm diameter stents. In general, for coronary stenting applications, dimensions will range between about 1.5 mm in diameter to about 4.0 mm in diameter, and generally unique sizes may be provided in a product line that vary by about 0.5 or 0.25 mm. In certain product offerings, the available sizes may range from about 2.0 or about 2.5 mm to about 4.0 mm. Moreover, lengths may also vary, in one regard may be between about 8 mm to about 40 mm, whereas typical frequent lengths for most standard coronary lesions are about 12 or about 18 mm, and for long stents about 24 mm or longer, possibly up to about 30 or 40 mm in length. Moreover, certain stent systems provide adjustable length for stenting vessels with a ratcheting stent delivery system. Various of the features herein disclosed may be incorporated into such system at each ratchet portion so as to accommodate the needs at the ends.

In another regard, various of the embodiments are herein described by reference to a metal stent chassis with a drug elution coating formed over the lattice structure of the stent chassis struts. However, other modes are contemplated and may benefit form the various embodiments herein described. For example, discrete wells or reservoirs of drug may be formed along the stent and elute therefrom. In another regard, the stent itself may be a drug eluting vehicle and not a two-part product with a stent and drug coating thereover. For example, certain bioerodable stents may be suitable for such embodiments and combined with the other embodiments herein described. In still a further regard, where drug elution coatings are used over stent scaffolding, such may be polymeric, non-polymeric, bioerodable, bioabsorbable, nanoporous, hydrogel, electroformed porous metal matrix, or other type of drug carrying and eluting coating modality as apparent to one of ordinary skill.

It is further contemplated that the particular arrangement, sizes, or other dimensions or relative shapes of components along the stents may vary from one size to the next. For example, where a 6 crown body design may be suitable for a particular size vessel, such as a 2.5 mm diameter vessel, a similar product made available in larger sizes such as 3.5 or 4.0 mm diameter may require more lattice scaffolding, and thus more crowns, to span the larger circumference with similar scaffolding results. These dimensions represent what are believed to be highly beneficial specific embodiments, but are not intended to be limiting to the broad aspects of the invention and dimensions may be modified according to one of ordinary skill in the art consistent with the objects and teachings provided throughout this disclosure.

Certain of the embodiments have been manufactured and are herein briefly shown and described by reference to certain photo's thereof for the purpose of providing the benefit of further illustration.

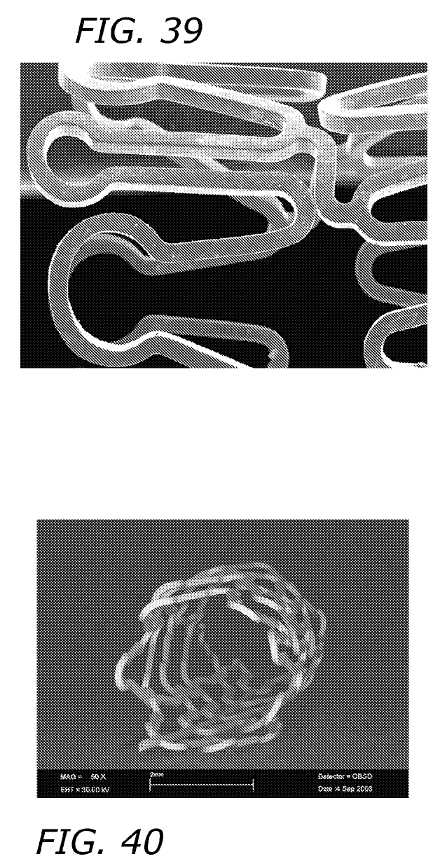

FIG. 39 shows an SEM picture of a side view along an end portion of a stent manufactured according to certain of the present embodiments with two alternating circumferential arrays of end-crowns having different sized bulb-shaped ends along the stent's end portion. As shown in this photo, the inter-crown distance between adjacent facing edges of the crown bulbs are substantially reduced, providing a more continuous pattern of drug elution there. Moreover, the shapes of the end crowns shown are substantially atraumatic to adjacent tissue due to their radius of curvature along the longitudinal axis of the stent.

FIG. 40 shows a picture of an end perspective view of a stent manufactured with a circumferential array of crowns with enlarged bulb-shaped enlargements, and is provided for the purpose of illustrating a shape that such bulbs take in the radially expanded condition after balloon expansion. More specifically, a radius of curvature is shown along the radial axis transverse to the longitudinal axis of the stent results. This is considered to be a well-suited structure for proper stent apposition in a curved vessel and relates to a further independently beneficial embodiment.

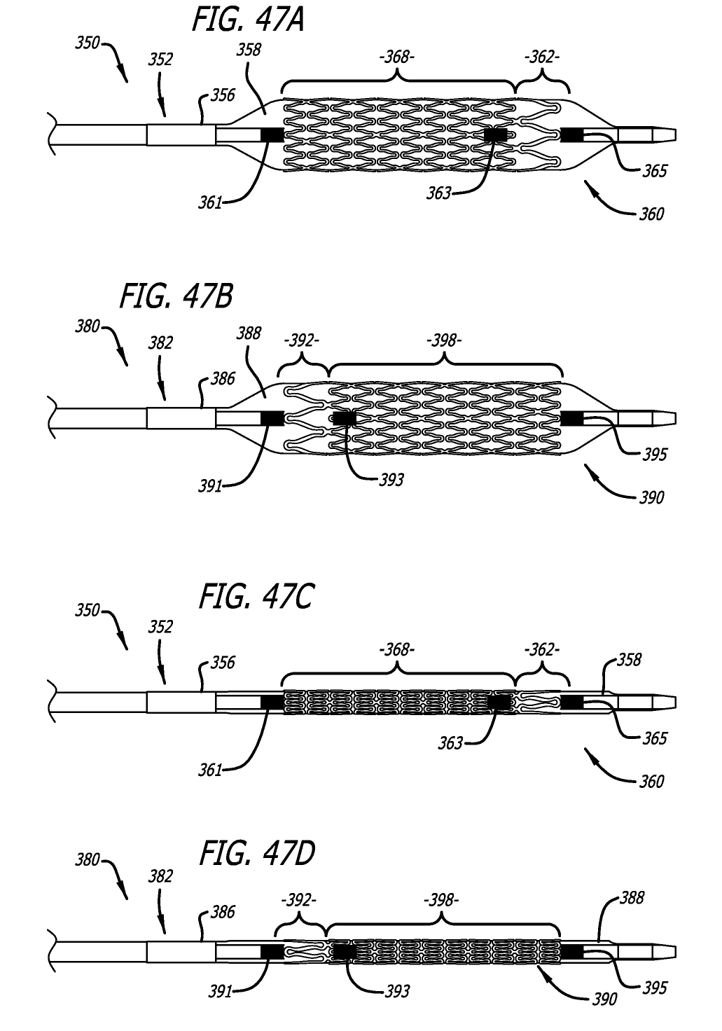

FIG. 47 depicts the overlapping stents and stent delivery system of FIG. 17 in expanded (FIG. 47A-B) and collapsed (crimped, FIG. 47C-D) configurations.

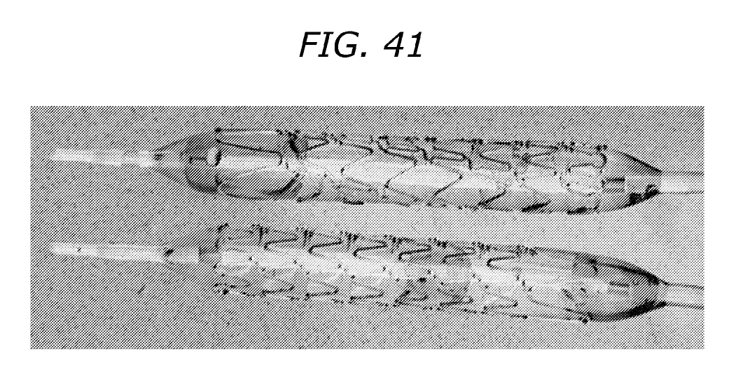

FIG. 41 shows a picture of two overlapping stents manufactured and assembled onto balloon catheters similar to the embodiments shown in FIGS. and 47, and includes proximal and distal overlapping ends on the two stents, respectively, which are each also opposite another end portion with a locally unique end crown structure providing beneficial long-term patency and drug dosing at that opposite end.

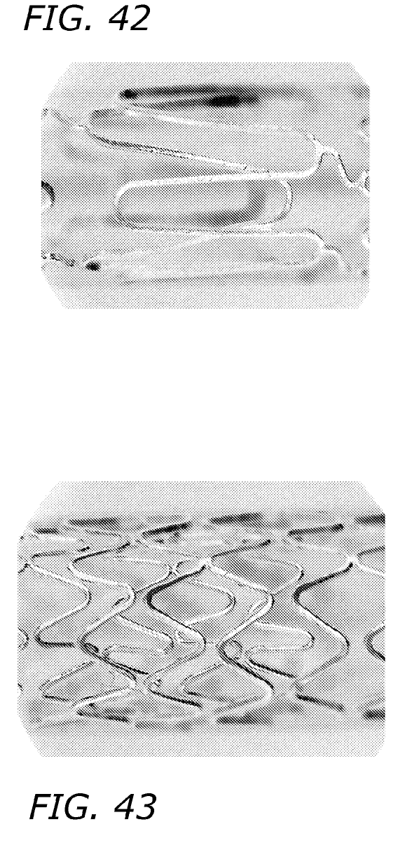

FIG. 42 shows a picture taken at 20× magnification under light microscopy of two overlapping stents after being manufactured and deployed in overlapping arrangement within a 3.0 mm lumen and under fluoroscopic guidance according to certain embodiments.

FIG. 43 shows a picture taken at 20× magnification under light microscopy of two commercially available stents in overlapping arrangement, and is provided for contextual purposes to illustrate the doubling of stent scaffolding metal in the overlap zone versus the more suitable structure resulting from the overlapping stents of FIG. 42.

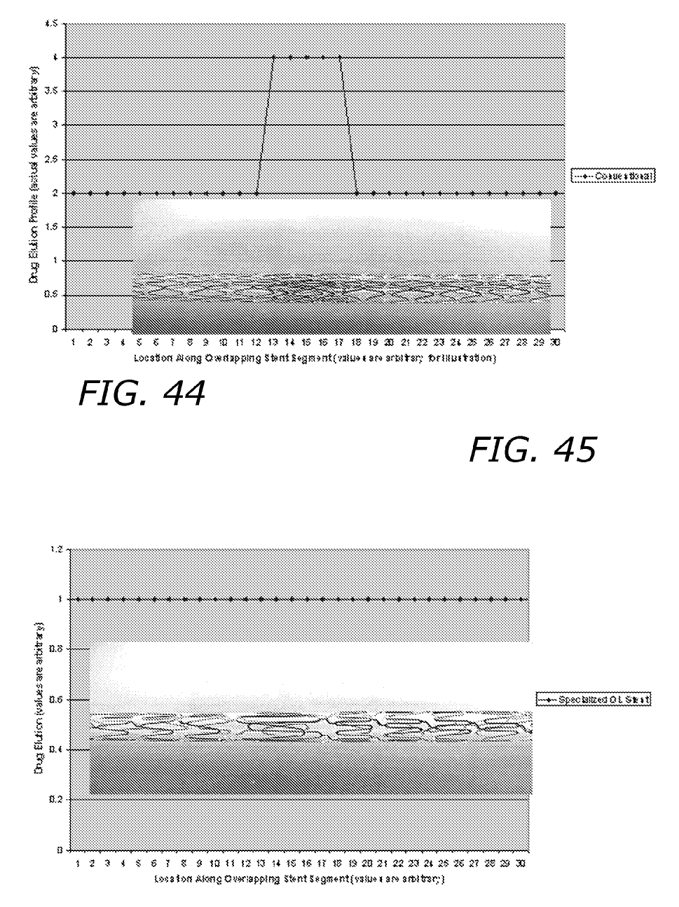

FIG. 44 shows a picture of two commercially available stents in overlapping arrangement. This picture has been modified with respect to the vertical/horizontal aspect ration so that it may be overlaid onto a graphical illustration of a drug elution profile expected from such overlapping arrangement in the context of adapting the overlapping stents for drug elution in conventional fashion. As shown, this drug elution is typically expected to be increased in the area of overlap, and generally by two-fold if there are no other modifications provided to accommodate for the overlapping arrangement.

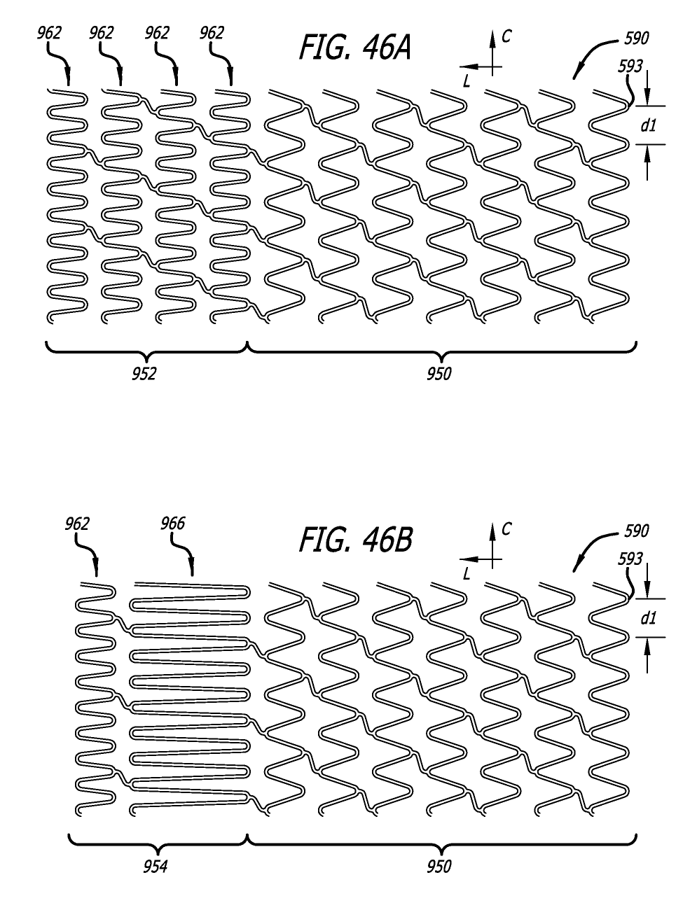

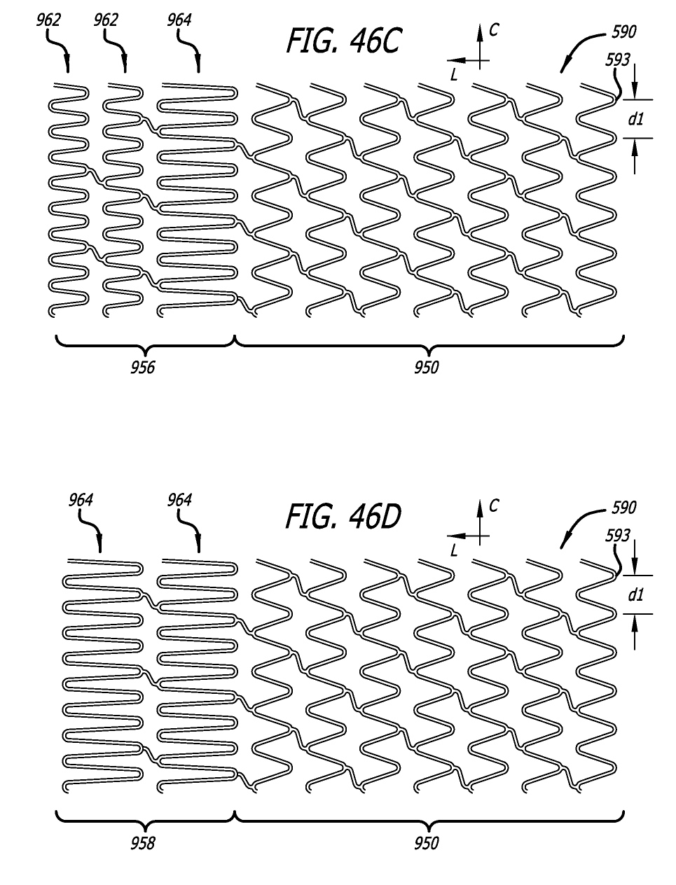

FIG. 45 shows a picture of two overlapping stents in overlapping arrangement according to one embodiment, and shows the picture overlaid onto a graphical illustration of a drug elution profile expected from such overlapping arrangement in the context of adapting the particular overlapping stents shown for drug elution also in conventional fashion. As shown, because the stent scaffold is reduced by 50 percent at the overlap zone for each stent, their doubling brings the drug-carrying capacity back to 100 percent, resulting in an expected drug elution profile that is substantially constant along the stented segment. It is further contemplated that though such is achieved with modifications in the drug-carrying stent scaffolding itself, other modes may be employed to reduce the potential doubling of drug delivery. For example, the coating formulation may be modified at the overlapping ends. Or, the amount of drug put onto a unit area of stent scaffolding there may be reduced. Such may be reduced by 50% for each overlapping end, or may be reduced to no elution on one overlapping end with no modification in elution at the other overlapping end. Each of these scenarios is exemplary of the various modes contemplated for achieving relative constant dosing along a stented vessel segment despite overlapping two drug eluting stents.

The various stent chasses herein shown and described also may be constructed according to various known structures, such as stainless steel, cobalt-chrome, polymeric scaffolds, or shape memory alloys such as nickel-titanium. Unless specifically indicated otherwise, such alternative constructions are contemplated for incorporation with and among the various embodiments herein shown and described to the extent appropriate to one of ordinary skill.

Although the description above contains many specifics, these should not be construed as limiting the scope of the disclosure but as merely providing illustrations of some of the presently preferred embodiments. Thus the scope of this stent system should be determined by the appended claims and their legal equivalents. Therefore, it will be appreciated that the scope of the stent system fully encompasses other embodiments which may become obvious to those skilled in the art, and that the scope of the stent system is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the stent system, for it to be encompassed by the present claims. Furthermore, no element, component, or method step in the present disclosure is intended to be dedicated to the public regardless of whether the element, component, or method step is explicitly recited in the claims. No claim element herein is to be construed under the provisions of 35 U.S.C. §112, sixth paragraph, unless the element is expressly recited using the phrase “means for.”

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Furthermore, numerous references have been made to patents and printed publications throughout this specification. Each of the above-cited references and printed publications are individually incorporated herein by reference in their entirety.

What is claimed is:

1. A stent assembly for implanting at least two stents at a location within a lumen, comprising:
first and second delivery systems each having a proximal end and a distal end that is adapted to be positioned at a location within a lumen;
first and second stents each with a first end portion, a second end portion and a central portion disposed between said respective first and second end portions;
said first end portion, said second end portion and said central portion defining a longitudinal axis along the length of each of said stents;
each of said first end portions having a first lattice structure that is different than a central lattice structure along the corresponding central portion;
said first stent is mounted on the distal end of said first delivery system with said first end portion located proximally of said second end portion;
said second stent is mounted on the distal end of said second delivery system with said first end portion located distally of said second end portion;
each of said first and second stents having a radially collapsed condition for delivery to said location;
wherein at said location each of the said first and second stents are adjustable from the respective radially collapsed condition to a radially expanded condition.

2. A stent assembly according to claim 1 wherein when expanded at said location, at least a part of said first end portions overlap.

3. A stent assembly according to claim 1 wherein said first lattice structures of each of said first end portions further have a lattice structure that is different than a second lattice structure along the corresponding second end portion.

4. A stent assembly according to claim 1 wherein said first lattice structure comprises a first circumferentially undulating pattern with a plurality of first strut segments wherein a circumferential array of M first end crowns is formed between adjacent first strut segments with said first undulating pattern having a first amplitude;
said second lattice structure comprises a second circumferentially undulating pattern with a plurality of second strut segments wherein said circumferential array of N second end crowns is formed between adjacent second strut segments with said second undulating pattern having a second amplitude; and
wherein said first and second amplitudes are different.

5. A stent assembly according to claim 4 wherein M is less than N.

6. A stent assembly according to claim 4 wherein M is 0.5 N.

7. A stent assembly according to claim 1 further comprising a bioactive agent in association with said first and said second stent wherein each of the first and second stents is adapted to exhibit a gradient of varied bioactive agent elution profiles along the respective stent length.

8. The stent assembly according to claim 7 wherein the gradient of varied bioactive agent elution profiles along the stent length comprises:
a first elution profile along the first end portion
a second elution profile along the central portion; and
a third elution profile along the second end portion that is different than the first and second elution profiles.

9. A stent assembly according to claim 7 wherein when expanded at said location, at least a part of said first end portions overlap to create an overlap zone and wherein the elution profile at said overlap zone is less than double the elution profile along the remaining portions of the stented segment.

10. A stent assembly according to claim 1 wherein the struts and crowns of said first end portion are of reduced thickness as compared to the struts and crowns of said second end portion or said central portion.

11. A stent assembly according to claim 1 further comprising:
a bioactive agent in association with said first stent and said second stent;
said respective first end portions are adapted to elute said bioactive agent according to a first elution profile;
said respective second end portions are adapted to elute said bioactive agent according to a second elution profile;
said respective central portions are adapted to elute said bioactive agent according to a third elution profile; and
wherein said first elution profile is substantially less than either the second or third elution profile.

12. A stent assembly according to claim 11 wherein said second and third elution profiles are substantially equivalent.

13. A stent assembly according to claim 1 wherein said first end, second end and central portions have the same longitudinal length.

14. A stent assembly according to claim 1 wherein said central portions have a longitudinal length that is greater than the longitudinal length of said first and said second end portions.

15. A stent assembly according to claim 1 wherein said first end portion has a longitudinal length that is about twice the longitudinal length of either of the second end portion or the central portion.

16. A stent assembly according to claim 1 wherein said first end portion comprises one or more elements and wherein the total longitudinal length of the first end portion does not exceed 4.0 mm.

17. A stent assembly according to claim 16 wherein each first end portion comprises up to four elements.

18. A stent assembly according to claim 16 wherein each element independently has a longitudinal length from about 0.5 mm to about 3.0 mm.

19. A stent assembly according to claim 1 wherein said first delivery system further includes a marker band at one or more locations selected from the group consisting of proximal to said first end portion, distal to said first end portion and distal to said second end portion.

20. A stent assembly according to claim 19 wherein said first delivery system includes a marker band at each of proximal to said first end portion, distal to said first end portion and distal to said second end portion.

21. A stent assembly according to claim 1 wherein said second delivery system further includes a marker band at one or more locations selected from the group consisting of distal to said first end portion, proximal to said first end portion and proximal to said second end portion.

22. A stent assembly according to claim 21 wherein said second delivery system further includes a marker band at each of distal to said first end portion, proximal to said first end portion and proximal to said second end portion.

23. A method for stenting a wall at a location within a lumen in a body of a patient comprising:
delivering a first stent to a first implant location within said lumen;
delivering a second stent to a second implant location within said lumen that overlaps with said first implant location such that confronting ends of said first and second stents overlap;
wherein the confronting end of each of said first and second stents comprise a lattice structure that is different than the lattice along the remaining portion of the respective stent.

24. The method of claim 23, further comprising overlapping said first and second stents in a manner such that their region of overlap does not have a substantially increased thickness profile off the wall of the lumen at said location.

25. The method of claim 23 wherein the longitudinal length of said overlap does not exceed 50% of the longitudinal length of either of the first stent or the second stent.

26. The method of claim 23 wherein the longitudinal length of said overlap is about 3.0 mm to about 5.0 mm.

27. A method for stenting a wall at a location within a lumen in a body of a patient comprising:
delivering a first stent to a first implant location within said lumen;
delivering a second stent to a second implant location within said lumen that overlaps with said first implant location such that confronting ends of said first and second stents overlap at an overlap zone; and
eluting a bioactive agent from said first and second stents such that an elution profile at said overlap zone is less than double an elution profile along the remaining portions of the stented segment.
